(19) United States
(12) Patent Application Publication
(10) Pub. No.: US 2025/0383323 A1
(43) Pub. Date: Dec. 18, 2025

(54) DEVICES AND METHODS FOR ULTRASOUND AND PHOTOACOUSTIC SCANNING

(71) Applicant: THE JOHNS HOPKINS UNIVERSITY, Baltimore, MD (US)

(72) Inventors: Jeeun KANG, Baltimore, MD (US); Emad BOCTOR, Ellicott City, MD (US); Hyunwoo SONG, Baltimore, MD (US)

(21) Appl. No.: 18/877,766

(22) PCT Filed: Jun. 23, 2023
(86) PCT No.: PCT/US2023/026066

§ 371 Date: Dec. 20, 2024

Related U.S. Application Data
(60) Provisional application No. 63/355,525, filed on Jun. 24, 2022.

(51) Int. Cl. G01N 29/24; G01N 29/06; G01N 29/265

(52) U.S. Cl. CPC G01N 29/2418; G01N 29/0654; G01N 29/265

(57) ABSTRACT

Methods and devices include an ultrasound and photoacoustic probe with a multiradius transducer, and a support with a primary axis. The multiradius transducer can include a proximal region, a distal region, and a transducer array. The proximal region can be adjacent the support, and the multiradius transducer can terminate at the distal region. In a first radius mode, the multiradius transducer can exhibit a first surface extending from the proximal region to a distal surface, the distal surface being in the distal region, the first surface characterized by a first radius. In a second radius mode, the multiradius transducer can exhibit a second surface extending from the proximal region to a second distal surface in the distal region, the second surface characterized by a second radius, where the second radius can be greater than the first radius.

### CROSS-REFERENCE TO RELATED APPLICATIONS

This application is the national stage entry of International Patent Application No. PCT/US2023/026066, filed on Jun. 23, 2023, and published as WO 2023/250132 A1 on Dec. 28, 2023, which claims the benefit of U.S. Provisional Patent Application No. 63/355,525, filed on Jun. 24, 2022, all of which are hereby incorporated by reference herein in their entireties.

### STATEMENT REGARDING FEDERALLY SPONSORED RESEARCH OR DEVELOPMENT

This invention was made with Government support under grant no. CA134675 awarded by the National Institutes of Health, grant no. 1653322 awarded by the National Science Foundation, and grant no. W81XWH-18-1-0188 awarded by the U.S. Army Medical Research and Development Command. The Government has certain rights in the invention.

### FIELD

Materials, components, and methods consistent with the present disclosure are directed to ultrasound and photoacoustic scanning.

### BACKGROUND

Transrectal ultrasound (TRUS) imaging is an effective clinical tool for cancer localization, biopsy guidance, and post-treatment surveillance (Perrin, 1992; Trabulsi et al., 2010). For several decades, 2-dimensional (2-D) TRUS imaging using a 1-D linear or curved array transducer has been a standard protocol, but it depends highly on the clinician's dexterity and subjective anatomic interpretation. Clinical urology reports the advantages of 3-D TRUS imaging, providing comprehensive anatomic context in prostate, renal and pelvic regions (Coleman et al., 2007), in which a 1-D linear array is inserted into rectal space through the longitudinal direction. The volumetric scanning is straightforwardly performed. Radio-frequency (RF) channel data for each radial plane is obtained with a single transmittance/reception event and repeats until filling the entire target volume using a motorized actuator (Fenster & Downey, 2000). Each pixel of the radial plane is beamformed using the RF channel data (usually by back-projection method), and then the image envelope is detected to generate a US image plane as a part of the volume. Once the volume is filled with US images at a scanning interval, internal voxels are interpolated to have a fixed unit pixel distance in the volume. Each radial plane has a certain slice thickness given a fixed elevation focusing lens of the 1-D linear array, defining radial spatial resolution. However, the radial spatial resolution is degraded as an imaging depth gets deeper due to lower scanline density and broader slice thickness, which is suboptimal to provide clear anatomical information to clinicians (M.-H. Bae & Jeong, 2000; S. Bae et al., 2018; Chang & Song, 2011).

Synthetic aperture focusing (SAF) techniques have been highlighted in the modern US imaging field for decades, which coherently compound time-multiplexed transmittance/reception events over sequential apertures at a specific target pixel to provide higher spatial resolution and enhanced texture uniformity (S. Bae et al., 2018). Most of the prior arts have focused on developing better imaging quality in the lateral direction (M.-H. Bae & Jeong, 2000; Chang & Song, 2011; Jensen et al., 2006; C. Kim et al., 2013). However, there have also been endeavors to effectuate the SAF technique in volumetric US imaging, necessitating a SAF that synthesizes multiple transmittance/reception events in an arbitrary direction (Andresen et al., 2010, 2011; Bottenus et al., 2016a; Kortbek et al., 2008; Nikolov & Jensen, 2000; Pedersen et al., 2007a). T. Lucas et al. presented an extended SAF technique to synthesize multiple cross-sections in different incident angles and positions for higher spatial and contrast resolution (Lucas et al., 2021). However, it requires a sophisticated wobbling scanning in an open imaging access point, which is inapplicable to the TRUS imaging setup. Intravascular US (IVUS) imaging was also tested with the SAF technique applied in the radial direction of the rotating element (rSAF), hoping to break through the limitation in spatial resolution defined by rotational scanning interval and focusing tightness. Such imaging setup is notably similar to that in the volumetric TRUS imaging. However, a recent investigation by S. Kang et al. concluded that the rSAF technique is ineffective with the IVUS imaging configuration (S. Kang et al., 2021). J. S. Kim et al. recently presented an rSAF-enhanced framework with a customized TRUS transducer (J. S. Kim et al., 2019). However, the progress has been stagnant primarily due to the lack of an analytical approach that enables a theoretical optimization of the TRUS imaging framework.

### SUMMARY

In one aspect, embodiments consistent with the present disclosure include a probe with a multiradius transducer, and a support comprising a primary axis, where the multiradius transducer further includes a proximal region, a distal region, and a transducer array. In an embodiment, the proximal region is adjacent the support, and the multiradius transducer terminates at the distal region. In a first radius mode, in an embodiment, the multiradius transducer exhibits a first surface extending from the proximal region to a distal surface, the distal surface being in the distal region. Further, the first surface is characterized by a length dimension approximately parallel to said primary axis, and the first surface is characterized by a first radius extending perpendicular to the length dimension. In an embodiment, and in first radius mode, the transducer array is situated on the first surface between the proximal region the distal surface. In a second radius mode, in an embodiment, the multiradius transducer exhibits a second surface extending from the proximal region to a second distal surface, the second distal surface being in the distal region. Further, the second surface is characterized by the length dimension approximately parallel to the primary axis, and the second surface is characterized by a second radius extending perpendicular to the length dimension. In an embodiment, and in second radius mode, the transducer array is situated on the second surface between the proximal region the second distal surface, and the second radius is greater than the first radius.

According to another exemplary embodiment of the present disclosure, a probe includes the probe of the previous embodiment, where the transducer array is configured for acoustic reception only.

In a further aspect, the probe is the probe of the first embodiment, where the probe is an ultrasound and photoacoustic probe, where the transducer array further includes a light fiber bundle, and where the transducer array is configured for ultrasound and/or photoacoustic imaging.

In an additional aspect, an ultrasound and photoacoustic probe of any of the previous embodiments can include external light and/or acoustic transmit systems that focally or broadly deliver the energy.

Further still, in another aspect, an embodiment can include an ultrasound and photoacoustic probe of any of the previous embodiments, where the multiradius transducer in either first radius mode or second radius mode is configured to scan a limited volume, where the limited volume is determined by the amount of light or acoustic energy received by the limited volume.

In a further aspect, an embodiment can include an ultrasound and photoacoustic probe of any of the previous embodiments, where the multiradius transducer identifies a plurality of positions in a scanning volume associated with a plurality of optical and/or acoustic energy transmittance values, the identification being based on acoustic data analysis or external tracking and position registration.

In an additional aspect, an embodiment can include an ultrasound and photoacoustic probe of any of the previous embodiments, where, when the multiradius transducer is in first radius mode, the transducer array situated on said first surface extends linearly along the first surface between said proximal region and said distal surface, and where, when the multiradius transducer is in second radius mode, the transducer array situated on the second surface extends linearly along the second surface between the proximal region and the distal region.

In a further aspect, an embodiment can include an ultrasound and photoacoustic probe of any of the previous embodiments, further including a second transducer array and a second light fiber bundle for ultrasound and/or photoacoustic imaging. In this embodiment, when the multiradius transducer is in first radius mode, the second transducer array is situated on the first surface between the transducer array and the distal surface, and, when the multiradius transducer is in second radius mode, the second transducer array is situated on the second surface between the transducer array and the distal region.

In another aspect, an embodiment can include an ultrasound and photoacoustic probe of any of the previous embodiments, where, when the multiradius transducer is in first radius mode, the transducer array situated on the first surface extends in a convex orientation at the first radius along the first surface between the proximal region and the distal surface. Furthermore, when the multiradius transducer is in second radius mode, the transducer array situated on the second surface extends in the convex orientation at the second radius along the second surface between the proximal region and the distal region.

Consistent with this disclosure, in an embodiment, the first surface of an ultrasound and photoacoustic probe can exhibit a generally cylindrical shape. Further still, the first distal surface can exhibit a hemispherical shape.

In a further aspect, the embodiment can include an ultrasound and photoacoustic probe, where the multiradius transducer further includes a multiaxis support, with the multiaxis support including a second axis. In this aspect, in the first radius mode, the second axis is approximately parallel with the primary axes and exhibits a first radial offset from the primary axis; and, in the second radius mode, the second axis is approximately parallel with the primary axis and exhibits a second radial offset from the primary axis. Consistent with this disclosure, the second radial offset can be greater than the first radial offset. Further still, the first radial offset can be approximately zero.

In a further aspect, the transducer array of any of the previous embodiments can be configured for rotational scanning.

In an additional aspect, a further embodiment can include ultrasound and photoacoustic probe that includes a multiradius transducer and a support including a primary axis. In an aspect, the multiradius transducer can include a proximal region, a distal region, and a transducer array and a light fiber bundle for ultrasound and/or photoacoustic imaging. Further still, the proximal region can be adjacent the support, and the multiradius transducer can terminate at the distal region. In an aspect, in a first radius mode, the multiradius transducer can exhibits a first surface extending from the proximal region to a first distal surface, the first distal surface being in the distal region, the first surface characterized by a length dimension approximately parallel to the primary axis and the first surface characterized by a first radius extending perpendicular to the length dimension. In first radius mode, the transducer array can be situated on said first distal surface. In an aspect, in a second radius mode, the multiradius transducer can exhibit a second surface extending from the proximal region to a second distal surface in the distal region, the second surface characterized by a second radius extending perpendicular to the length dimension. In an embodiment, the transducer array is situated on the second distal surface, and the second radius is greater than the first radius.

In a further embodiment, a method of ultrasound and photoacoustic scanning consistent with the current disclosure can include scanning a target volume using the ultrasound and photoacoustic probe of any of the previous embodiments to acquire target volume data. In an aspect, the target volume can include a plurality of volumes, and the scanning can include repetitively scanning the plurality of volumes.

Further still, in an aspect, a method of ultrasound and photoacoustic scanning can include any of the previous methods, where the scanning can include rotating the ultrasound and photoacoustic probe generally about the primary axis.

Further still, in an embodiment, a method of ultrasound and photoacoustic scanning consistent with this disclosure can include any of the previous methods, and further include identifying and rejecting a plurality of grating lobe artifacts in the acquired target data.

Further still, in another aspect, an embodiment can include an ultrasound and photoacoustic probe of any of the previous embodiments, where the multiradius transducer in either first radius mode or second radius mode is configured to scan a limited volume, where a maximal limit of the second radius is determined by the image or pressure sensor measurement to prevent any complications due to too much radius extension.

Additional features and embodiments of the invention will be set forth in part in the description which follows, and in part will be obvious from the description, or may be learned by practice of the invention. It is to be understood that both the foregoing general description and the following detailed description are exemplary and explanatory only and are not restrictive of the claimed subject matter.

### BRIEF DESCRIPTION OF THE DRAWINGS

The accompanying drawings, which are incorporated in and constitute a part of this specification, illustrate exemplary embodiments and together with the description, serve to explain the principles of the disclosure. In the figures:

FIGS. 1 and 2 depict a transducer device consistent with the current disclosure.

FIGS. 3 and 4 depict models of a TRUS-rSAF method consistent with the current disclosure.

FIG. 5 depicts a method of improving image quality consistent with the current disclosure.

FIGS. 6 and 7 depict a transducer device consistent with the current disclosure in an exemplary use.

FIGS. 8-10 depict data associated with critical parameter optimization of a model consistent with the current disclosure.

FIG. 11 depict aspects of imaging performance and simulation consistent with the current disclosure.

FIG. 12 depicts a further transducer device and results of quantitative measurement consistent with the current disclosure.

FIGS. 13-15 depict further aspects of imaging performance, simulation, and phantom experiment consistent with the current disclosure.

### DESCRIPTION OF THE EMBODIMENTS

Reference will now be made in detail to the disclosed embodiments of the disclosure, examples of which are illustrated in the accompanying drawings. Wherever possible, the same reference numbers will be used throughout the drawings to refer to the same or like parts.

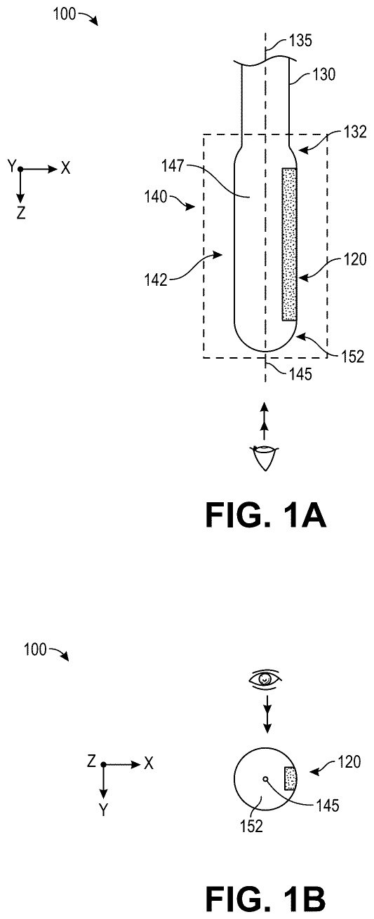

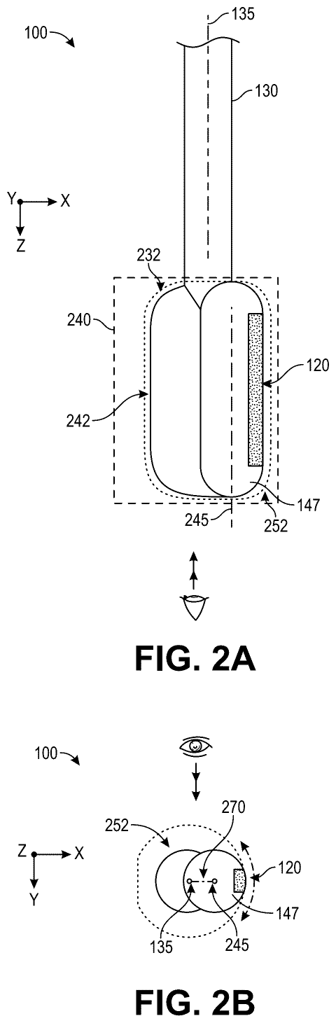

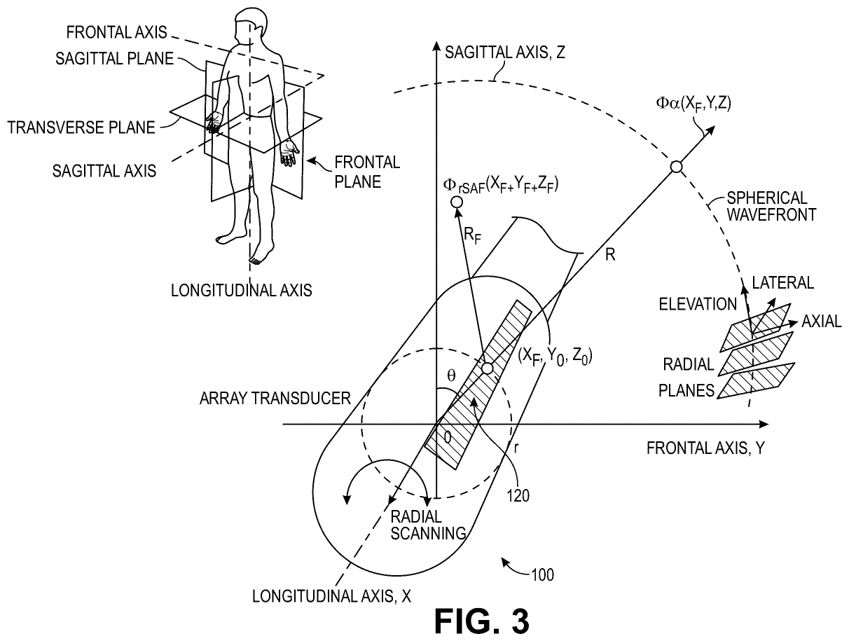

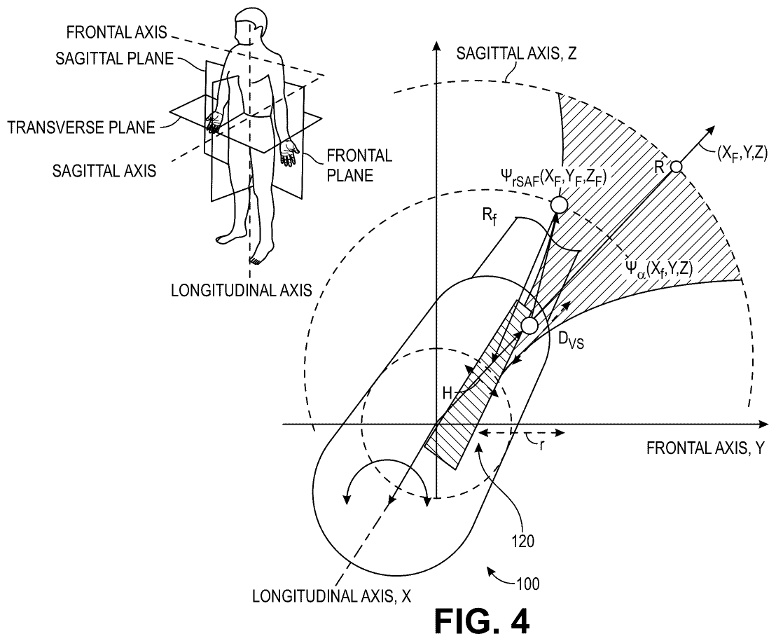

FIGS. 1 and 2 depict a transformable transducer device 100 consistent with the current disclosure. FIGS. 1 and 2 each depict two perspectives, labeled 1A and 2A, respectively (such that the “y-axis” is perpendicular to the image plane), and 1B and 2B, respectively (such that the “z-axis” is perpendicular to the image plane). In FIG. 1, transducer device 100 is shown in first radius mode, and in FIG. 2 transducer device 100 is depicted in second radius mode.

With reference to FIG. 1, transducer device 100 includes support 130 and multiradius transducer 140 in first radius mode. Support 130 includes primary axis 135, and multiradius transducer 140 includes multiaxis support 147, which is characterized by second axis 145. Multiradius transducer 140 can include proximal region 132 and distal region 152. In certain embodiments, distal region 152 can include a distal surface, which can be a generally hemispherical shape. The proximal region 132 is adjacent support 130, and multiradius transducer 140 terminates in the distal region 152. As shown in FIG. 1, transducer array 120 is configured as a linear array along the surface of the multiradius transducer 140 in region 142 between the proximal region 132 and the distal region 152.

With reference to FIG. 2, transducer device 100 again includes support 130 which includes primary axis 135. As depicted in FIG. 2, however, in certain embodiments, transducer device 100 can include multiradius transducer 240 in second radius mode. Consistent with certain embodiments, multiradius transducer 240 can include proximal region 232 and distal region 252, and again includes multiaxis support 147. In FIG. 2, however, multiaxis support 147 includes second axis 245. The proximal region 232 is adjacent support 130, and multiradius transducer 240 terminates in the distal region 252. As shown in FIG. 2, transducer array 120 is configured as a linear array along the surface of the multiradius transducer 240 in region 242 between the proximal region 232 and the distal region 252. In second radius mode, in certain embodiments, the second axis 245 of multiaxis support 147 is offset by a distance 270 (shown in view 2B) from the primary axis 135. Consistent with the disclosure, transducer device 100 can be configured specifically for TRUS and TRPA imaging. Further still, transducer device 100 can be configured for rotational scanning.

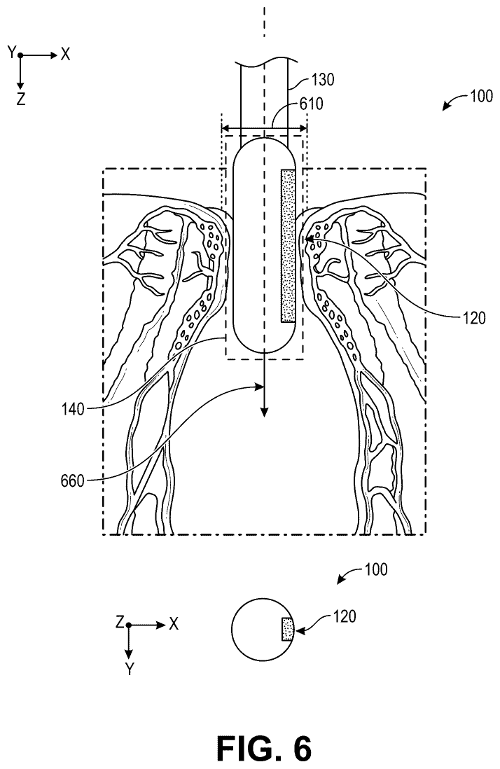

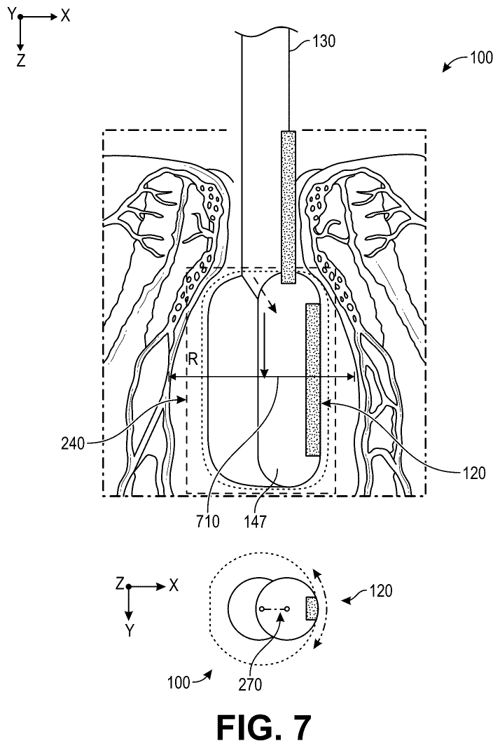

As depicted in FIGS. 6 and 7, when transducer device 100 is being inserted in a cavity space 660 for use, such as through an anus towards rectum, transducer device 100 may be maintained in first radius mode (shown in FIGS. 1 and 6) to maximally alleviate the patients' pain or discomfort during the insertion. Once the multiradius transducer 140 is in the cavity space 660 (such as a rectal space), the multiradius transducer 140 in first radius mode can be configured to expand to multiradius transducer 240 in second radius mode, as depicted in FIGS. 2 and 7. For example, multiradius transducer 140 may be configured to expand its radius to maximally utilize the given diameter 710 with rectum wider than anus entry (3-5 cm width 710 in rectum vs. 2-3 cm width 610 at anus). In order to estimate the cavity space 660 available for multiradius transducer 140 and 240—which may vary patient-by-patient—prior to placement of multiradius transducer 140 in first radius mode, the cavity space 660 may be filled with an air or liquid balloon with sensors to measure the available space. Such sensors can include image and/or pressure sensors. One of ordinary skill in the art should appreciate that expansion of the probe, if excessive, can present complications, such as tissue damage. Accordingly, to prevent complications due to too much radius extension, the sensors, such as image and/or pressure sensors, can be utilized to limit expansion of the maximal second radius.

One of ordinary skill in the art should appreciate that the expansion of multiradius transducer 140 in first radius mode to multiradius transducer 240 in second radius mode may be accomplished using a variety of techniques, all of which are consistent with the current disclosure. For example, consistent with the disclosure, multiaxis support 147 may consist of a relatively rigid apparatus that supports transducer array 120. Multiradius transducers 140 and 240 can include a flexible surface portion capable of maintaining a variable volume of fluid, such that the multiradius transducer 140 in first radius mode assumes the volume of a first volume of fluid contained by the flexible surface portion, and multiradius transducer 140 in second radius mode assumes the volume of a second volume of fluid contained by the flexible surface portion, where the second volume of fluid is greater than the first volume of fluid. The entry and exit of fluid volume from multiradius transducer 140 and 240 can be accomplished through support 130 (not shown). Consistent with certain embodiments, the flexible surface portion of multiradius transducer 140 and 240 can be configured to connect with multiaxis support 147 such that the transducer array 120 is maintained at the surface portion, and the surface of the entire multiradius transducer 140 and 240 assumes a generally symmetric relation to primary axis 135 and is configured for rotational scanning.

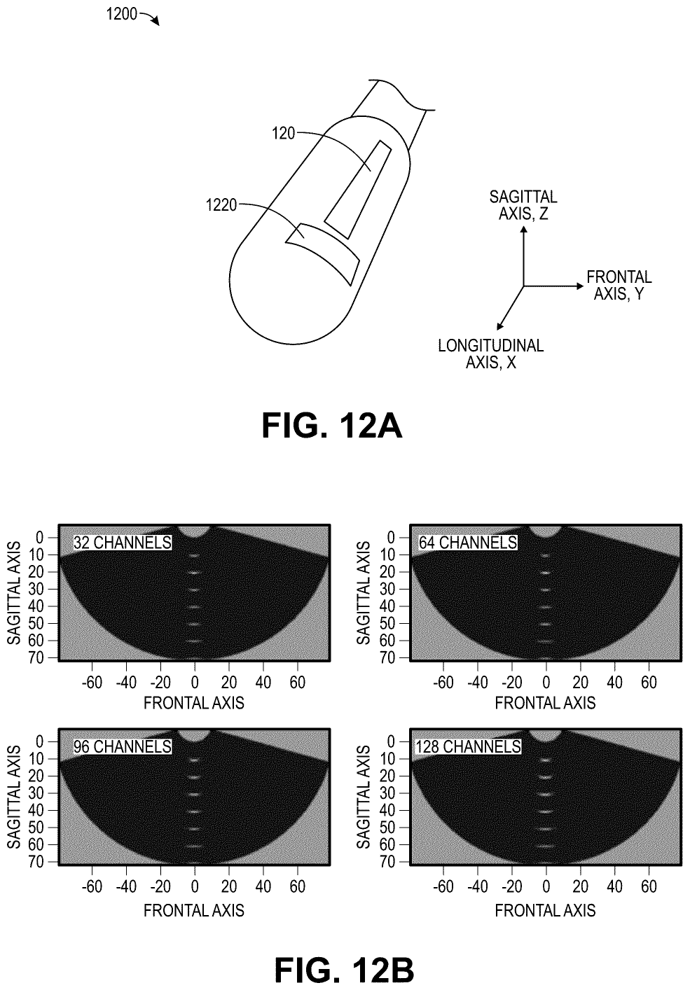

One of ordinary skill in the art should also appreciate that the transducer array 120 depicted in FIGS. 1, 2, 6, and 7 can be in a different orientation. For example, consistent with a microconvex array, a transducer array can be shortened and broadened as, for example, depicted by transducer array 1220 in FIG. 12 (but without transducer array 120 as shown in FIG. 12). For example, transducer array 1220 is depicted as extending in a convex orientation on the surface of the multiradius transducer (as a microconvex array 1220). Alternatively, as depicted in FIG. 12, a pair of transducer arrays may be implemented on a transducer device consistent with the current disclosure as a bi-planar array (including both linear transducer array 120 and microconvex transducer array 1220 as shown in FIG. 12).

Further still, a convex transducer array may be situated entirely in the distal region of a multiradius transducer device consistent with the current disclosure. In such a manner, such a convex transducer may be configured to exhibit a plurality of convex arcs consistent with a plurality of radius modes.

Moreover, consistent with the current disclosure, a transducer device may be configured as shown in FIG. 1, 2, 6, 7, or 12, but without an integrated light emitting or delivery component and/or acoustic transmitting components, where a transducer array is used only for acoustic reception.

Alternatively, or in addition, a transducer device may be configured as described herein, where an external light and/or acoustic transmit systems focally or broadly deliver energy.

Further still, consistent with the current disclosure, a transducer device may be configured as described above, but configured such that the multiradius transducer scans only the volume that receives an effective amount of light or acoustic energy either in the first or second radius modes. One of ordinary skill in the art would appreciate that differing extents of effective optical and/or acoustic energy transmittance can be determined based on acoustic data analysis or external tracking and position registration.

Moreover, methods consistent with the current disclosure can include obtaining scanning data using any of the transducer devices described herein, where the multiradius transducer, either in the first or second radius modes, is used to repetitively scan different volumes in order to complete a target volume.

One of ordinary skill in the art will appreciate that scanning data obtained from the device consistent with the current disclosure can exhibit grating lobe artifacts. Accordingly, portions of this disclosure below provide methods for identifying and rejecting grating lobe artifacts in the data, among other improvements.

### Implementation

In this section, a model of the TRUS-rSAF method is established based on two coordinate frames: {sagittal (z), longitudinal (x), frontal (y)} axes to define the global Cartesian coordination of imaging FOV; {axial, lateral, elevation} axes define the local Cartesian coordination relative to the TRUS linear array. Based on the axes, the frontal-sagittal plane is defined as a transverse plane, longitudinal-sagittal plane as a sagittal plane, and frontal-longitudinal plane as a frontal plane. The radial axis is global coordination, combining sagittal and frontal axes. The mathematical derivation is established on the global Cartesian coordination system unless mentioned otherwise.

### Field Analysis Model of TRUS-rSAF Imaging

FIG. 3 depicts a 2-D theoretical field analysis model of TRUS-rSAF method in transverse plane (frontal-sagittal axis) at specific longitudinal position x_{f} of the linear TRUS array. The dot labelled Φ_{rSAF}(x_{f}, y_{f}, z_{f}) indicates the synthetic focusing pixel (x_{f}, y_{f}, z_{f}); (x_{f}, y₀, z₀) is an element position; and θ is a scanning angle in the transverse plane. Left-top image shows the global coordination of the theoretical model. One can omit the x axis in the model for a more straightforward representation. The acoustic source rotates along with the origin with a rotating radius

$$r = \sqrt{{y}_{o}^{2} + {z}_{0}^{2}} .$$

In the figure, the acoustic wave propagates along with a scanning angle θ=sin⁻¹ α with respect to the sagittal axis. The velocity potential Φ of the monochromatic spherical wave can be expressed at an observation point (y,z) as

$${Φ}_{α} ( y , z , t ) = \frac{{e}^{- j ω t}}{j λ {‖ R ‖}_{2}} {Ψ}_{α} ( y , z ) , \qquad ( 1 )$$

where ω, λ and ψ_{α} represent the angular frequency of the transmitted acoustic wave, wavelength, and transmit beam pattern, respectively. The continuous transmit beam pattern at a depth of R can be expressed as

$${Ψ}_{α} ( y , z ) = {e}^{jkR} = {e}^{jk \sqrt{{( y - r α )}^{2} + {( z - r β )}^{2}}} \qquad ( 2 )$$

where k=2π/λ represents the wavenumber and β=cos θ.

In the defined coordination, the transmit beam is synthesized by compounding multiple acoustic waves propagating with different radial scanning angles by adjusting the synthetic time delays τ(α) to be coherently focused on a desired focal point in the transverse plane at x_{f}, (y_{f},z_{f}). Thus, the resultant beam pattern can be expressed as

$${Ψ}_{rSAF} ( {y}_{f} , {z}_{f} ) = {c}_{0} {\int }_{- ∞}^{∞} {p}_{s} ( α ) τ ( α ) {Ψ}_{α} ( y , z ) d α \qquad ( 3 )$$

where

$${c}_{0} = \frac{1}{j λ {‖ R ‖}_{2}} , τ ( α )$$

represents ure synthetic time delay function and pₛ(α) denotes the effective radial synthetic window over the range of α used in the TRUS-rSAF imaging. In certain embodiments, a technological benefit of the TRUS-rSAF method can come from the transmit beam synthesis, while the receive beam pattern will be identical to that of the TRUS-REF method, where a single transmittance/reception event is used to reconstruct a radial plane. Therefore, the analytical development here will focus on describing the synthesized transmit beam. When the beams are focused at (y_{f}, z_{f}) in a specific longitudinal position x_{f}, the synthetic time delay function is

$$τ ( α ) = {e}^{- {jkR}_{f}} = {e}^{- jk \sqrt{{( {y}_{f} - r α )}^{2} + {( {z}_{f} - r β )}^{2}}} . \qquad ( 4 )$$

Hence, substituting Eqs. 2 and 4 to Eq. 3 defines the synthetic transmit beam pattern of the TRUS-rSAF imaging as follows:

$${Ψ}_{rSAF} ( {y}_{f} , {z}_{f} ) = {c}_{0} {\int }_{- ∞}^{∞} {p}_{s} ( α ) {e}^{jk ( R - {R}_{f} )} d α . \qquad ( 5 )$$

By using the Fresnel approximation, R−R_{f} can be reduced to

$$R - {R}_{f} = \frac{{y}^{2} - {y}_{f}^{2}}{2 {z}_{f}} + \frac{r ( y - {y}_{f} )}{{z}_{f}} α . \qquad ( 6 )$$

The Fresnel approximation has been frequently used in the biomedical ultrasound field (Cobbold, 2006) to neglect the non-zero y position of the US element by being considerably smaller than the imaging depth in a target scanline in the axial direction, z_{f}. Such approximation is usually valid in biomedical ultrasound to image deep tissue with a small US array footprint. Therefore, substituting Eq. 6 with Eq. 5 yields the final beam profile.

$${Ψ}_{rSAF} ( {y}_{f} , {z}_{f} ) = {c}_{0} {e}^{jk \frac{{y}^{2} - {y}_{f}^{2}}{2 {z}_{f}}} {\int }_{- ∞}^{∞} {p}_{s} ( α ) {e}^{- jk \frac{r ( y - {y}_{f} )}{{z}_{f}} α} d α = {c}_{0} {e}^{jk \frac{{y}^{2} - {y}_{f}^{2}}{2 {z}_{f}}} {ℱ [ {p}_{s} ( α ) ]}_{{f}_{y} = \frac{{ry}^{′}}{λ {z}_{f}}} \qquad ( 7 )$$

where y′=y−y_{f}. In the equation, the synthetic transmit beam pattern is represented by the Fourier transform ( [symbol] [·]) of the radial synthetic window function pₛ(α). When assuming the α range as [−αₘₐₓ/2, αₘₐₓ/2] and uniform element directivity in the radial field without apodization, pₛ(α), can be expressed as

$${p}_{s} ( α ) = rect ( \frac{α}{{α}_{max}} ) . \qquad ( 8 )$$

The resultant synthetic transmit beam pattern of the TRUS-rSAF imaging is acquired by substituting Eq. 8 to Eq. 7:

$${Ψ}_{rSAF} ( {y}_{f} , {z}_{f} ) = {c}_{1} sin c ( \frac{r {α}_{max}}{λ {z}_{f}} {y}^{′} ) , \qquad ( 9 )$$

where

$${c}_{1} = {c}_{0} \cdot {α}_{max} {e}^{jk \frac{{y}^{2} - {y}_{f}^{2}}{2 {z}_{f}}} ,$$

and the null-to-null mainlobe width (i.e., y_{ML}′) is defined by

$${y}_{ML}^{′} = \frac{λ {z}_{f}}{r {α}_{max}} . \qquad ( 10 )$$

Eq. 10 confirms that the spatial resolution of the TRUS-rSAF imaging is proportional to acoustic wavelength (i.e., the fundamental frequency of TRUS transducer) and imaging depth while being inversely proportional to rotation radius r and radial synthetic window defined by αₘₐₓ. The grating lobe is not considered due to a continuous synthetic window pₛ(α).

Consistent with this disclosure, the disclosed model is further practicalized using discrete radial scanning angles, as each angle necessitates an individual transmittance/reception event rather than having a continuous aperture in the α domain as in Eq. 8. Here, consider N transmit angles uniformly discretized with an interval Δα, which can be equated by

$${p}_{s}^{′} ( α ) = {\sum }_{n = 0}^{N - 1} δ ( α - {α}_{n} ) , \qquad ( 11 )$$

where αₙ=Δα·n−αₘₐₓ/2 and Δα=αₘₐₓ/(N−1). Consequently, the synthetic transmit beam pattern of the TRUS-rSAF imaging can be converted to discretized form as

$${Ψ}_{s} ( {y}_{f} , {z}_{f} ) = {c}_{0} {e}^{jk \frac{{y}^{2} - {y}_{f}^{2}}{2 {z}_{f}}} {\sum }_{n = 0}^{N - 1} {e}^{- jk \frac{r {α}_{n}}{{z}_{f}} y} = {c}_{1}^{′} \frac{sin π \frac{rN Δα}{λ {z}_{f}} y}{sin π \frac{r Δα}{λ {z}_{f}} y} , \qquad ( 12 )$$

where

$${c}_{1}^{′} = {c}_{0} \cdot {e}^{\frac{{y}^{2} - {y}_{f}^{2} + r {α}_{max}}{2 {z}_{f}} y} .$$

The null-to-null main lobe width is given from (12):

$${y}_{{s}_{ML}}^{′} = \frac{λ {z}_{f}}{rN Δα} = \frac{λ {z}_{f}}{r {α}_{max}} . \qquad ( 13 - 1 )$$

The discretized sampling in α domain results in grating lobes in the beam field positioned at

$${y}_{{s}_{GL}}^{′} = \frac{λ {z}_{f}}{r Δα} n ( n = 1 , 2 , 3 , … ) . \qquad ( 13 - 2 )$$

Therefore, consistent with the current disclosure, an analytical solution of the TRUS-rSAF imaging has shown that critical yet flexible parameters are r and Δα, defining theoretical spatial resolution and grating lobe positions. Consistent with the disclosure, one can perform a heuristic optimization on those critical parameters in the following Section. For λ, we will follow the well-established specification in the TRUS-REF imaging.

### Implementation Using Virtual Sources

Consistent with the disclosure, one can implement the TRUS-rSAF model synthesizing radial wavefronts diverging from virtual sources (VSs) at a fixed lens focusing point of each radial plane at a specific axial distance d_{VS}. This approach resembles the concept that has been used for the SAF technique in the lateral direction with electrical focusing (Frazier & Jr., 1998).

FIG. 4 depicts implantation of TRUS-rSAF method in 2-D transverse plane at a specific longitudinal position x_{f}. The bolded parameters dᵥₛ, h, and r (adjacent the dotted arrows in FIG. 4) are the variables in our rSAF optimization. The solid arrow extending from Ψ_{rSAF}(x_{f}, y_{f}, z_{f}) (labelled Rf), extending to Ψ_{rSAF}(x_{f}, y_{f}, z_{f}) (from the solid circle labelled dᵥₛ), and extending between these two arrows in FIG. 4 indicate the synthetic time delay τ(α) of the given radial transmit beam Ψ_{α}(y, z) to reconstruct the target focusing point (y_{f}, z_{f}). Accordingly, FIG. 4. defines the revised model of the TRUS-rSAF method. At the VS of each radial plane, the acoustic wavefront forms two-way focusing for transmission and reception, while other depth regions will have a spherical wavefront. The wavefront from each VS can be synthesized by adjusting the time-of-flight passing through the corresponding VS to the target pixel (y_{f}, z_{f}) in the transverse plane at a specific longitudinal position x_{f}. Here, the primary consideration for the SAF with a 1-D element should be to count only the transmittance planes providing adequate overlap at the target focal point (Bottenus et al., 2016b; Pedersen et al., 2007b). In synthetic focusing of each radial plane, the radial acoustic wavefront first travels from (y₀, z₀) to the VS with the distance of d_{VS}. From the VS, the acoustic wavefront travels to the focusing point (y_{f}, z_{f}), and we denoted this distance as d_{τf}(i, z) at the ith dataset within the synthetic window. Therefore, the distance in the total transmittance pathway is dₜ(i, z)=d_{VS}+d_{tf}(i, z), which results in a transmit time-of-flight, τₜ(i, z)=dₜ(i, z)/c, where c is a constant speed of sound in biological tissue (i.e., 1,540 m/sec). On the other hand, an acoustic reception distance dr is defined as the shortest pathway reversing from (y_{f}, z_{f}) to element in the TRUS transducer (y₀, z₀), leading to receiving time-of-flight τᵣ(z)=dᵣ(z)/c (i.e., R_{f}). In all, adjacent radial planes can be synthesized to the target pixel (y_{f}, z_{f}) by compensating the focusing delay at each synthetic angle, τ_{f}(i, z)=τₜ(i, z)+τᵣ(z). The coherent synthesis of effective radial planes in a target imaging slice can be equated by

$${I}_{rSAF} ( {θ}_{n} , z ) = \frac{1}{{N}_{syn} ( z )} {\sum }_{i = n - {N}_{syn} ( {θ}_{n} , z ) / 2 + 1}^{n + {N}_{syn} ( {θ}_{n} , z ) / 2} {I}_{i} ( {θ}_{n} , z ) , \qquad ( 14 )$$

where I_{rSAF}(θₙ, z) is a signal intensity in a target radial plane at θₙ, and z of imaging angle and depth, respectively; N_{syn}(θₙ, z) is the number of effective radial planes for the TRUS-rSAF imaging, defined at specific target radial angle θₙ and imaging depth z by their overlap of adjacent beam profiles; and Īᵢ(θₙ, z) is the US intensity at the focal point (θₙ, Z) from ith dataset within the synthetic window which is delay-compensated by τ_{f}(i, z). The model is valid for each imaging angle θₙ (n=1, 2, . . . , N), comprising entire imaging volume in an interval of Δθ (i.e.,

$$Δθ = {sin}^{- 1} Δα ) .$$

### Simulation and Optimization

### Spatial Resolution-Oriented Optimization Strategy

Eq. 13-1 suggests the critical parameters to define the spatial resolution of the TRUS-rSAF imaging: acoustic wavelength λ, radial synthetic window defined by αₘₐₓ, and probe radius r. In practice, the element height h and d_{VS} determines αₘₐₓ, and r determines the overlap among adjacent radial planes. Here, we define the optimal TRUS transducer for the effective rSAF technique with a specific design objective to maximize the spatial resolution. A heuristic optimization was performed on the critical parameters. Basic design specifications followed those of a clinical TRUS transducer (linear array in BPL9-5/55, BK Ultrasound, Inc., MA, USA): 6.5 MHz, center frequency; fractional bandwidth, 80%; 5 mm, elevation aperture size h; 20 mm, elevation focusing depth d_{VS}; 10 mm, probe radius r. Imaging parameters were as follows: 0.4724°, rotation interval in degree Δθ; 128 or 280 radial scanlines to compose the radial fields-of-view (FOVs) of [−30°, 30°] and [−66.14°, 66.14°], respectively. The target imaging depth of interest was from 0 mm to 70 mm. Consistent with the disclosure, one can choose Δθ to scan the deep tissue region at a good interval. The Δθ at 0.4724° is designed to have 14 scanlines in a single radial FWHM at deep imaging depth (6.43 mm at 70-mm depth) while providing clinically relevant temporal resolution (˜6 volume per second). During the optimization, clinically relevant ranges for the critical parameters were evaluated to secure their practicality in clinics: d_{VS}={5, 10, 15, 20, 25} mm; h={3, 4, 5, 6, 7} mm; and r={5, 10, 15} mm. The optimal d_{VS} was first defined for subsequent evaluation of h and r. Note that sound propagation speed c was fixed at 1,540 m/s during the optimization.

N_{syn}(θₙ, z) was determined for each combination of critical parameters to maximize an effective overlap among adjacent beam profiles along the volumetric scanning trajectory in the radial direction. This approach enables us to find the peak spatial resolution by synthesizing only the effective radial transmission/reception events for a target focal point. Using Field-II simulation of wire targets in an imaging depth range from 10 mm to 70 mm at 10-mm intervals, we tested each combinational setup while increasing N_{syn} (i.e., 3, 5, 7, . . . ) until finding the narrowest radial full-width-half-maximum (FWHM) at a depth of interest (Jensen, n.d.; Jensen & Svendsen, 1992). A geometrical beam profile model was configured using h, z_{VS}, and N_{syn} was derived at the deepest imaging depth, 70 mm (i.e., N̅_̅{̅s̅y̅n̅}̅). The model finally defined N_{syn} (θₙ, z), giving the corresponding optimal @max at individual depths z (i.e., αₘₐₓ(z)=Δα·N_{syn}(θₙ, z)). The FWHM at each depth was evaluated.

Consistent with the disclosure, one can evaluate the impact of spatial resolution-oriented optimization on the signal-to-noise ratio (SNR) in the canonical definition:

$$SNR = 20 {log}_{10} \frac{E [ {I}^{2} ( {θ}_{n} , z ) ]}{{σ}^{2}} , \qquad ( 15 )$$

where E[·] is the expectation of the signal amplitude, and σ is the noise power. The definition delivers acoustic power difference between signal and background noise power during data acquisition. A short d_{VS} may secure high αₘₐₓ but result in lower acoustic power density in deep tissue than the clinical TRUS-REF imaging, which deteriorates resultant signal sensitivity. On the other hand, the coherent frame compounding in the TRUS-rSAF framework will bring a counter effect enhancing the signal-to-noise amplitude ratio theoretically with a factor of \sqrt{N_{syn}(θₙ, z)} if approximating that Iᵢ(θₙ, z)=Īᵢ(θₙ, z) as in Eq. 14. The SNR presents the projected signal sensitivity due to these opposing effects.

The SNR at each combinational parameter setup was evaluated in Field-II imaging simulation. A transmit beam profile was first analyzed in each setup to quantify the acoustic energy over imaging depth. We also simulated wire targets located at 10-mm intervals from 10 mm to 70 mm to define the signal component. In addition, a randomized Gaussian noise image at −20-dB mean intensity was separately simulated in the corresponding imaging FOV to define the noise component. The SNR deviation due to the optimized TRUS-rSAF method was calculated compared to its negative control, named TRUS-CON method here, where imaging is performed with exact specifications as in the optimized TRUS-rSAF method but comprising a target volume with only the center radial plane at each scanning angle. This setup is to omit the benefit of the coherent radial synthesis intentionally. The TRUS-REF method uses a clinical TRUS array transducer (BPL9-5/55 in this study) to represent a clinical performance expected in modern clinics.

Contrast and information entropy contrast (IEC) can be measured in the tissue-mimicking Field-II simulation data to compare the TRUS-REF and TRUS-rSAF imaging performance. Contrast represents deviation between hyperechoic and cyst regions and IEC evaluates spatial acuity quantified by microstructural entropy (Hu et al., 2008; Ju et al., 2015; Lee et al., 2012; Tsui et al., 2017), which are defined as

$$Contrast = 10 {log}_{10} \frac{{\sum }_{y , z ⊆ HR} {I}^{2} ( y , z )}{{\sum }_{y , z ⊆ CR} {I}^{2} ( y , z )} , \qquad ( 16 )$$

$$IEC = InEn \cdot \hat{C} \qquad ( 17 )$$

where HR and CR are hyperechoic mass and anechoic cyst regions, respectively. InEn and Ĉ are an information entropy and a mean contrast, respectively, within the regions-of-interest (ROIs), which are defined by

$$InEn = - {\sum }_{i = {\hat{I}}_{min}}^{{\hat{I}}_{max}} Prob ( {\hat{I}}_{i} ) \cdot {log}_{2} [ Prob ( {\hat{I}}_{i} ) ] , \qquad ( 17 - 1 )$$

$$\hat{C} = \frac{1}{( {K}_{y} - 1 ) ( {K}_{z} - 1 )} {\sum }_{z = 0}^{{K}_{z} - 2} \vert pxl ( y , z ) - pxl ( y , z + 1 ) \vert , \qquad ( 17 - 2 )$$

where, Îₘᵢₙ, Îₘₐₓ are the minimum and maximum grayscale pixel intensities, Prob (Îᵢ) is the probability of pixel distribution at ith gray level, and pxl(y, z) is the pixel intensity at (y, z) coordinate (i.e., frontal and sagittal axes, respectively). Contrast is a practical image quality metric that contains multi-factor influences during image acquisition and reconstruction, including the acoustic power, sidelobe artifacts, and grating lobe artifacts at a target ROI.

### Critical Parameters in TRUS-rSAF Transducer Optimization

### Impact of d_{VS}

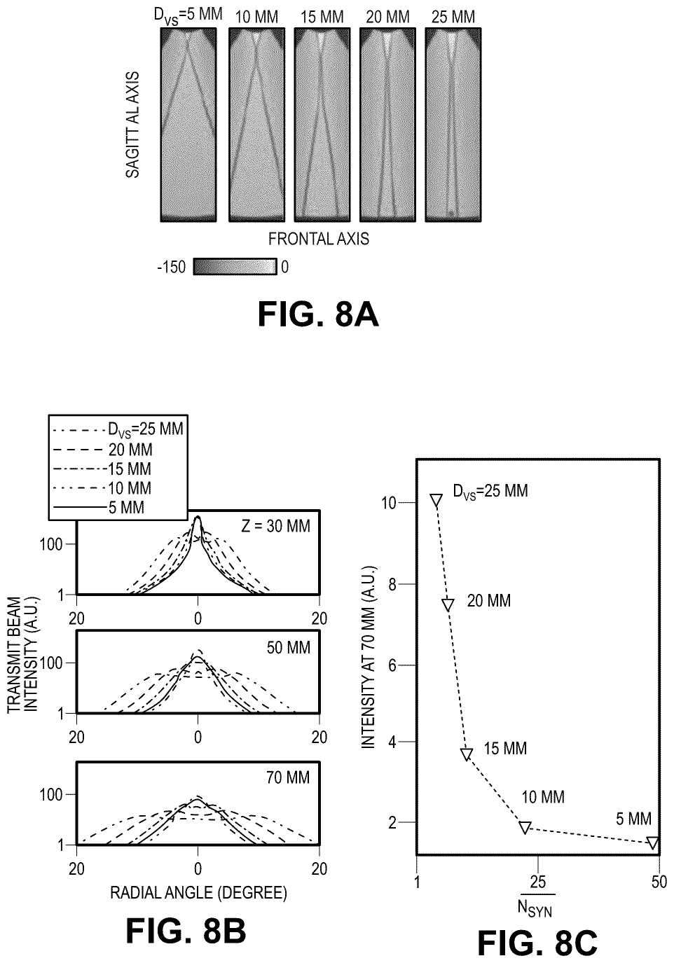

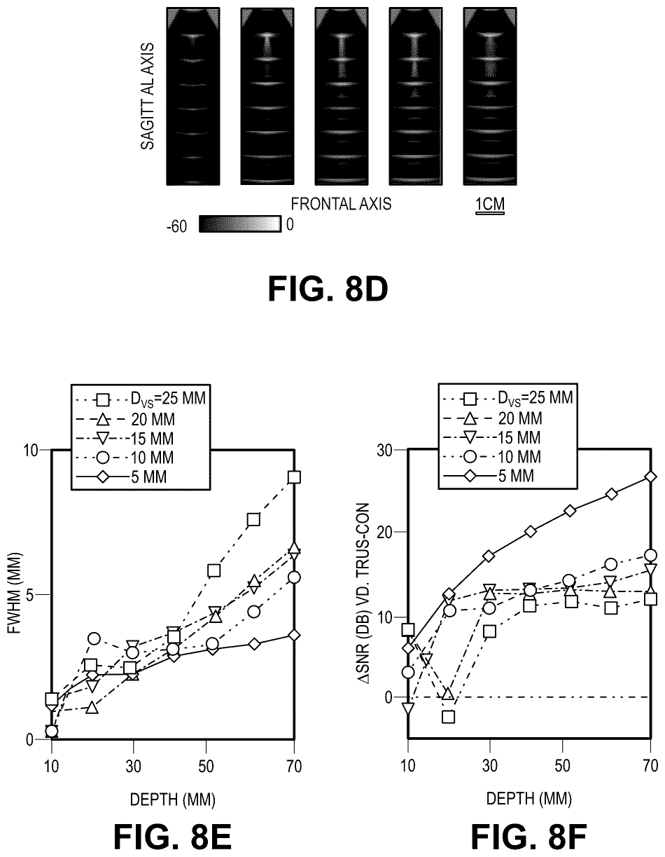

FIG. 8 depicts critical parameter optimization on d_{VS} impact on TRUS-rSAF imaging performance. (a) Transmit beam profiles at different d_{VS}. The lines in FIG. 8a indicate-6-dB contour from each depth. Intensity was normalized to the maximum when d_{VS}=25 mm. (b) Transmit beam intensity at 30-, 50-, and 70-mm depths. (c) Beam intensity as a function of N̅_̅{̅s̅y̅n̅}̅ at different d_{VS} on the observation point in (a) (where the observation point is shown in the far right figure in 8(a) as a dot). (d) Field-II wire-target simulation data at different d_{VS}. (e) Full-width-half-maximum (FWHM) over imaging depth. (f) SNR difference between the TRUS-rSAF and TRUS-CON imaging over imaging depth.

Consistent with the current disclosure, the transmit beam profile of the TRUS array was first analyzed as a function of d_{VS}. 1-D radial intensity profiles were extracted at {30, 50, 70} mm using the Field-II simulation of transmit beam pattern (FIG. 8a). The transmit beam profiles become weaker as d_{VS} gets shorter, sacrificing the signal sensitivity at a deep depth. For example, d_{VS}={5, 10, 15, 20} mm provided {92.75, 79.68, 47.24, 13.50} % and {90.25, 76.40, 62.84, 29.69} % of reductions of maximal acoustic power reaching to at 30- and 70-mm depths, respectively, when compared to the case with d_{VS} at 25 mm (FIG. 8b). Such changes will affect the signal component for SNR calculation. On the other hand, a transmit beam profile had wider beam distribution when the shorter d_{VS} was applied, which resulted in a greater N̅_̅{̅s̅y̅n̅}̅: {49, 23, 11, 7, 5} (i.e., θₘₐₓ={22.67°, 10.39°, 4.72°, 2.83°, 1.89°}) when d_{VS}={5, 10, 15, 20, 25} mm, respectively. Therefore, a net SNR will be determined by a competing effect of transmit power and synthetic window width specified by d_{VS}, as shown in FIG. 8c. FIG. 8d shows the 2-D TRUS-rSAF images reconstructed in the transverse plane. The TRUS-rSAF imaging enhanced spatial details in the visual assessment when the shorter d_{VS} was applied. The FWHM measurement at each depth quantitatively validated the trend (FIG. 8e). For example, at 70-mm depth, the FWHM of the target was {3.66, 5.66, 6.37, 6.66, 9.10} mm at d_{VS}={5, 10, 15, 20, 25} mm, respectively.

It should be noted that the SNR definition in Eq. 15 requires equal attention to noise components, which should be related to the coherent compounding effect with N_{syn}(θₙ, z). FIG. 8f shows the SNR difference between the TRUS-CON and TRUS-rSAF imaging with different d_{VS}. It was notable that more significant SNR improvements were obtained in the TRUS-rSAF imaging at deeper imaging depth with shorter d_{VS}. For example, {6.28, 17.13, 22.66, 26.60}-dB higher SNRs were achieved at {10, 30, 50, 70}-mm depths when d_{VS}=5 mm, whereas there were only {9.14, 8.01, 11.58, 11.80}-dB improvements when d_{VS}=25 mm.

Note that there was no further analysis on the grating lobe with d_{VS} because no impact is expected given with Eq. 13-2.

### Impact of h

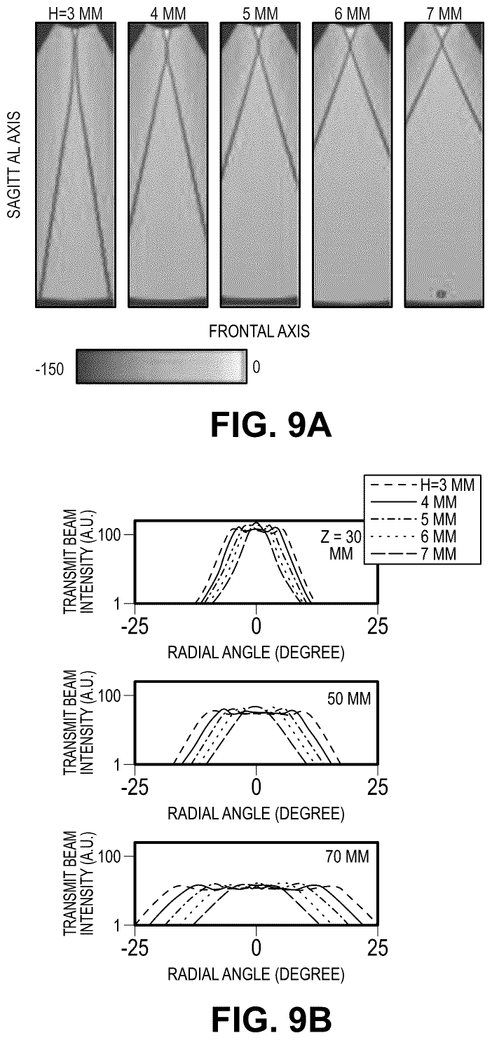

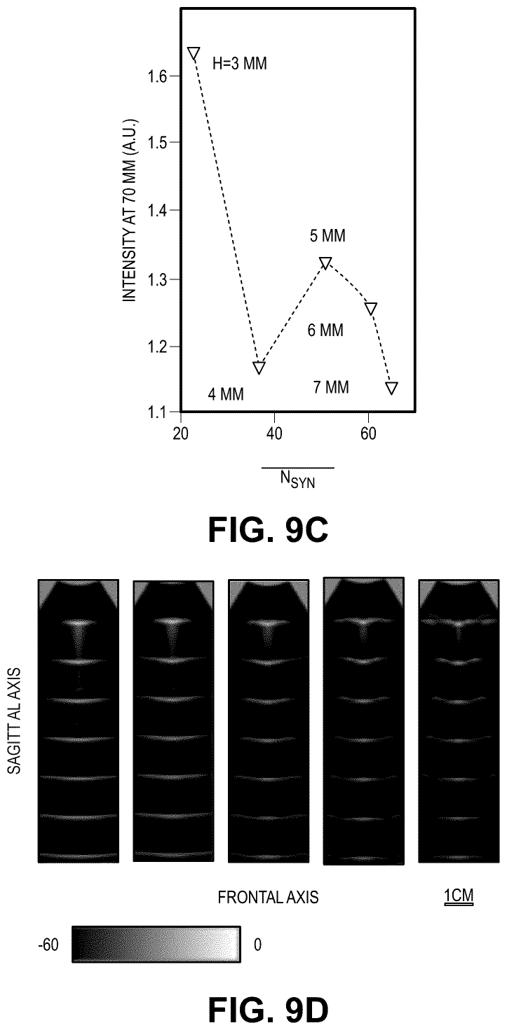

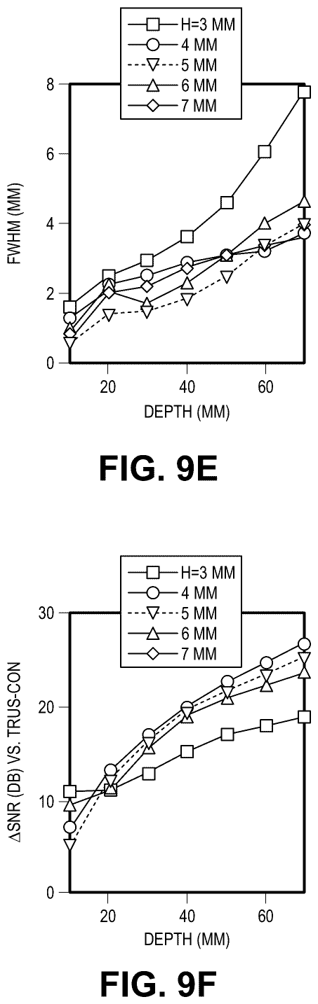

FIG. 9 depicts critical parameter optimization on h impact on TRUS-rSAF imaging performance. (a) Transmit beam profiles at different h. The lines in FIG. 9a indicate −6-dB contour from each depth. Intensity was normalized to the maximum when h=3 mm. (b) Transmit beam intensity at 30-, 50-, and 70-mm depths. (c) Beam intensity as a function of N̅_̅{̅s̅y̅n̅}̅ at different h on the observation point in (a) (where the observation point is shown in the far right figure in 9(a) as a dot). (d) Field-II wire-target simulation data at different h. (e) Full-width-half-maximum (FWHM) over imaging depth. (f) SNR difference between the TRUS-rSAF and TRUS-CON imaging over imaging depth.

FIG. 9a shows the transmit beam profiles at different h={3, 4, 5, 6, 7} mm. Note that the d_{VS} is fixed at 5 mm based on the previous optimization, and other parameters were used as given in the clinical reference TRUS transducer. For a fair comparison, the total transmit acoustic power for different h were not equalized. The acoustic power when h={4, 5, 6, 7} mm was reduced by {12.64, 21.51, 24.24, 30.74} %, {6.95, 14.65, 16.21, 23.37} %, and {0.61, 3.03, 12.73, 16.97} % at 30-, 50-, and 70-mm depths, respectively, when compared to the corresponding cases of having h=3 mm (FIG. 9b). Notably, the intensity changes among h={4, 5, 6, 7} mm were not simply inversely proportional given by the dependency on the different transmit aperture sizes (FIG. 9c). A net SNR at each depth will be determined by combining the transmit power and N_{syn}(θₙ, z). One might expect an increase in signal sensitivity proportional to h, giving a wider aperture to detect acoustic signals. However, h is a dominant factor to define the transmit beam divergence from the VS, and more divergency with wider h will lower the sensitivity in the out-of-VS depth range.

The spatial resolution was evaluated for the corresponding setups using the Field-II simulation data of wire targets (FIG. 9d). The N̅_̅{̅s̅y̅n̅}̅ was proportional to h: {23, 37, 51, 61, 65} for h={3, 4, 5, 6, 7} mm, which was predictable with the divergence of focused wavefront proportional to the transmit aperture width (FIG. 9b). The FWHMs measured at each depth indicated the inversely proportional relationship between FWHM and h (FIG. 9e). For example, the FWHMs of the target at 30 mm are {2.92, 2.50, 2.17, 1.68, 1.48} mm for h={3, 4, 5, 6, 7} mm, respectively. At 70-mm depth, the FWHMs became {7.76, 4.61, 3.77, 3.66, 3.92} mm for h={3, 4, 5, 6, 7} mm, respectively. Therefore, the larger h will provide a more consistent and higher spatial resolution.

FIG. 9f shows the SNR of the TRUS-CON and TRUS-rSAF imaging with different h. The higher SNRs were obtained when applied the TRUS-rSAF imaging with wider h. Representatively, {5.104, 16.12, 21.59, 25.26}-dB higher SNRs were achieved at {10, 30, 50, 70}-mm depths when h=7 mm, whereas there were only {10.73, 12.84, 16.95, 18.95}-dB improvements when h=3 mm. Therefore, the change in h affects SNR combinatorially by acoustic divergence and total acoustic power transmittance. There was no further analysis on the grating lobe with h as no impact is expected with Eq. 13-2.

### Impact of r

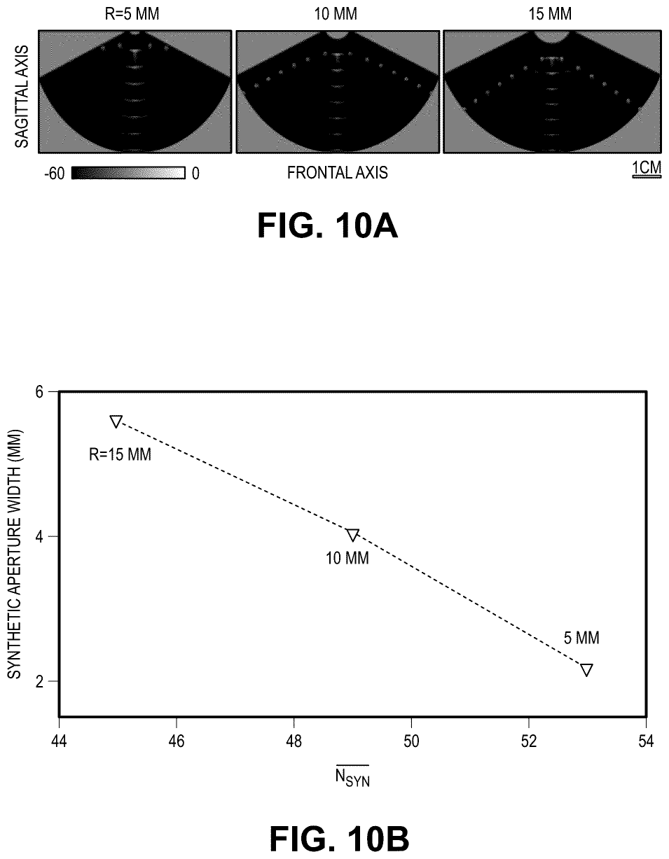

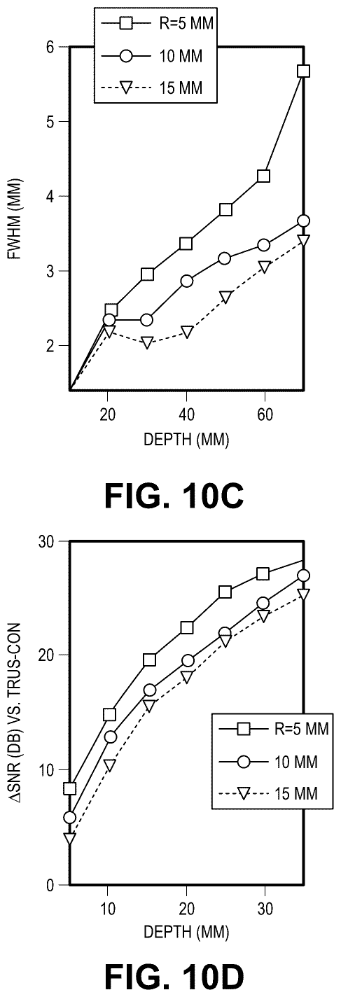

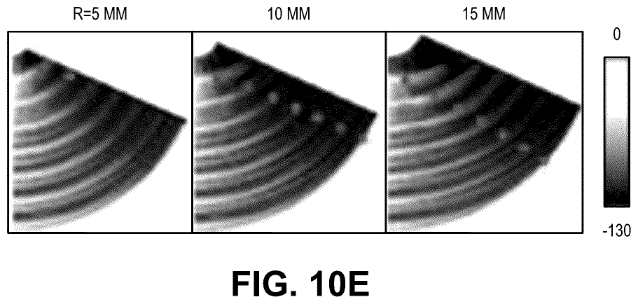

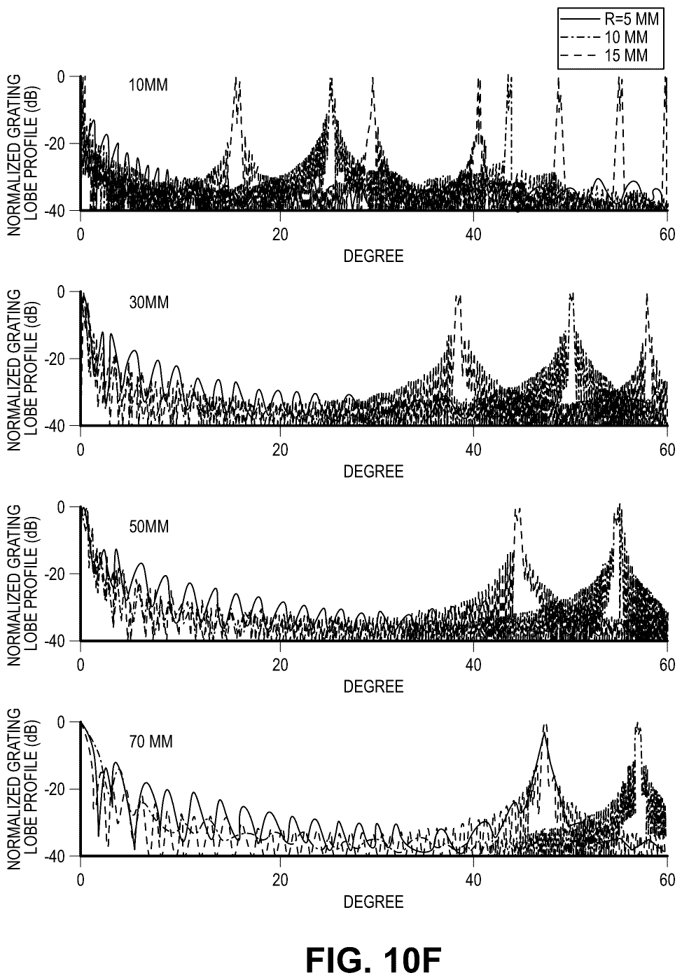

FIG. 10 depicts critical parameter optimization on r impact on TRUS-rSAF imaging performance. (a) Field-II wire-target simulation data at different r. (b) Aperture size as a function of given N̅_̅{̅s̅y̅n̅}̅. (c) Full-width-half-maximum (FWHM) over imaging depth. (d) SNR difference between the TRUS-rSAF and TRUS-CON imaging over imaging depth. (e) TRUS-rSAF images with extended dynamic range. The dots in FIG. 10(e) indicate the first grating lobe positions. (f) Theoretical grating lobe positions at 10-, 30-, 50-, and 70-mm depths and at different r.

Consistent with the current disclosure, the analytical solution has shown that r is the most critical parameter affecting spatial resolution and grating lobe positions. A heuristic optimization was performed by testing a range of r: {5, 10, 15} mm (FIG. 10a). As predicted in Eq. 13, the measured FWHMs of the wire target at each depth presented the inversely proportional relationship between FWHM and r as shown in FIG. 10c. FWHMs at 30-, 50-, and 70-mm depths indicated {2.94, 2.34, 2.01} mm, {3.81, 3.16, 2.64} mm, and {5.63, 3.66, 3.38} mm when r is {5, 10, 15} mm, respectively. It can be explained by an interesting observation that the N̅_̅{̅s̅y̅n̅}̅ is inversely proportional to r when fixed Δθ at 0.4724°: {53 49, 45} for r={5, 10, 15} mm, leading to the corresponding radial aperture sizes at {2.18, 4.04, 5.57} mm, respectively (FIG. 10b).

SNR was also calculated at each r. Note that r will not affect the individual radial plane, so the SNR improvements should be determined by N_{syn}(θn, z). FIG. 10d presents the SNR proportional to N̅_̅{̅s̅y̅n̅}̅. For example, 28.39-, 26.87-, 26.26-dB SNR improvements were obtained at 70-mm depth when r at {5, 10, 15} mm, respectively.

Another crucial impact of r on the positions of grating lobes is also considered. The proximity of the first grating lobes to the main lobe at the imaging axis is regarded as inversely proportional to the imaging contrast. The analytic solution in Eq. 13-2 suggests that the enlarged r shifts the grating lobes toward the main lobe. B-mode images with extended dynamic range (FIG. 10e) validate the theoretical expectation matched with theoretical synthetic transmit beam patterns in FIG. 10f. The theoretical first grating lobe positions when r={5, 10, 15} mm were at #{43.78, 25.63, 15.99}°, + {64.03, 50.11, 38.62} °, + {66.97, 55.19, 44.88} °, and ={68.10, 57.30, 47.67}° at 10, 30, 50, and 70-mm depths, respectively. The spatial sampling at the VS depth can also explain these results. The sampling intervals in the wavelength unit are {0.35, 0.52, 0.69} λ when r={5, 10, 15}, which confirms coarser sampling with larger r, bringing more grating lobe artifacts into a scanning angle. Therefore, the TRUS-rSAF imaging with a larger r results in more grating lobe artifacts. The following section of practical optimization will include the strategy to suppress the grating lobe artifacts.

### Implementation

In previous sections, an analytical approach was established to design the critical parameters for a TRUS transducer enabling the effective volumetric rSAF technique: d_{VS}=5 mm, h=7 mm, and r=15 mm. This section presents an integrative optimization workflow of the TRUS-rSAF imaging with a specific design criterion to outperform the TRUS-REF imaging that illustrates a basic expectation in clinics.

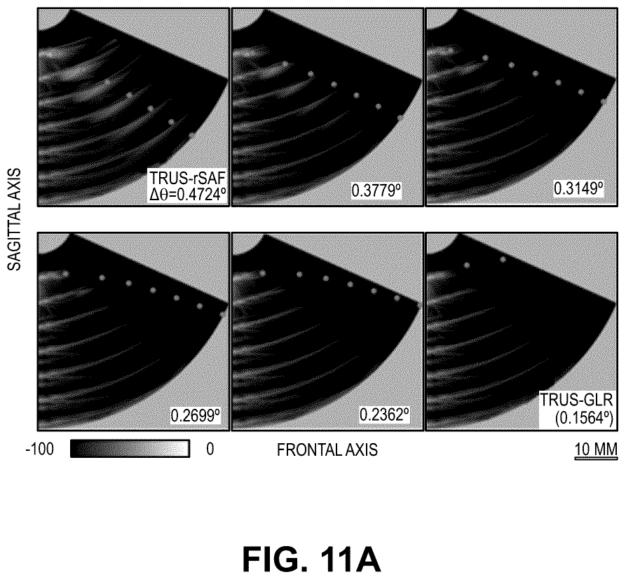

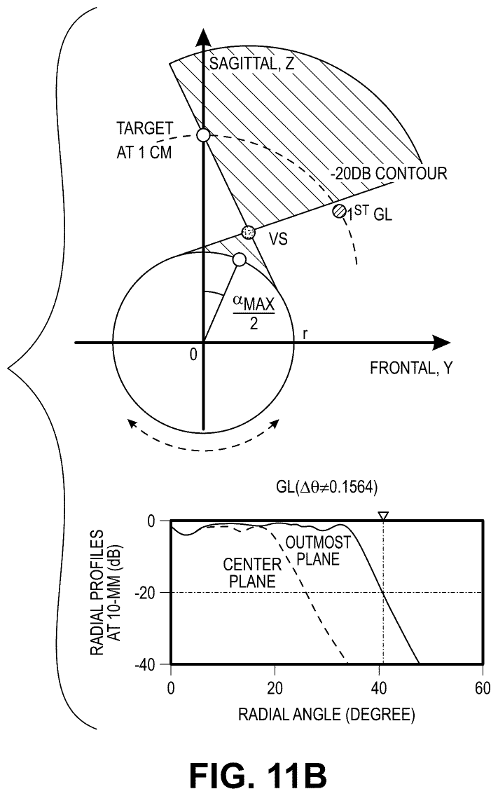

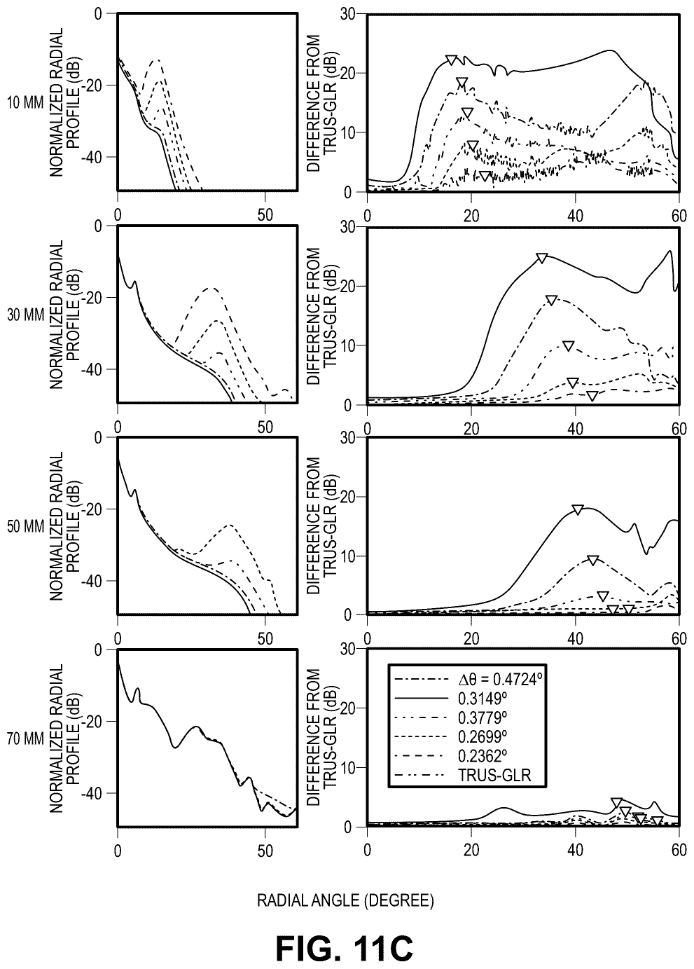

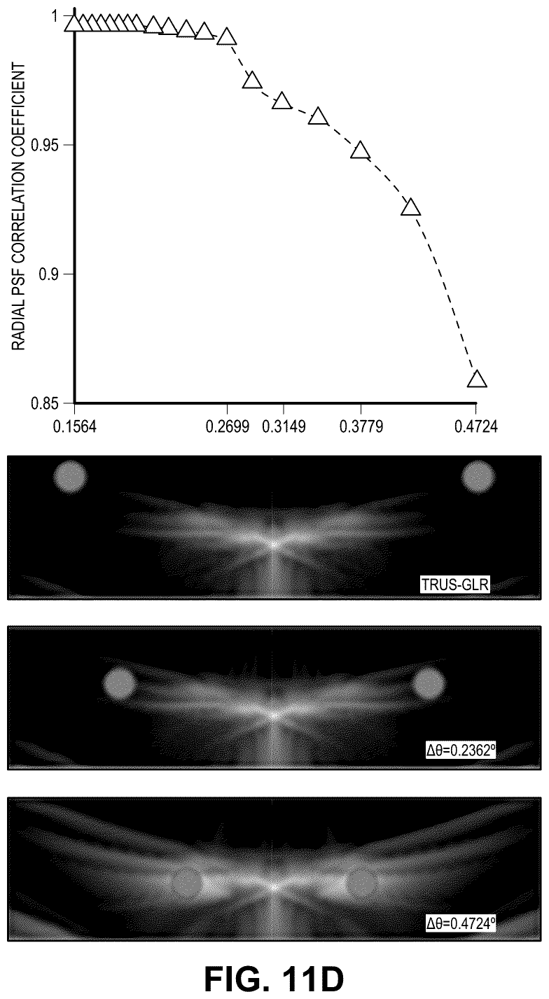

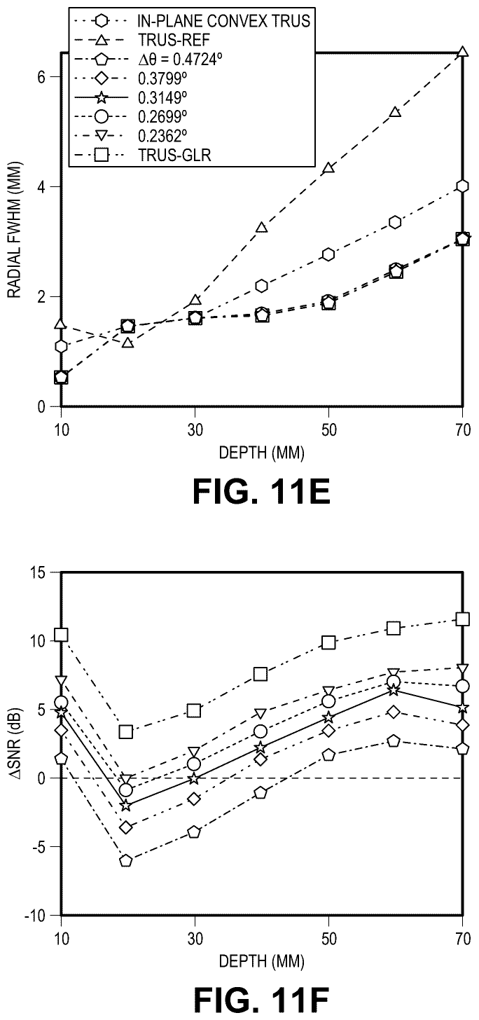

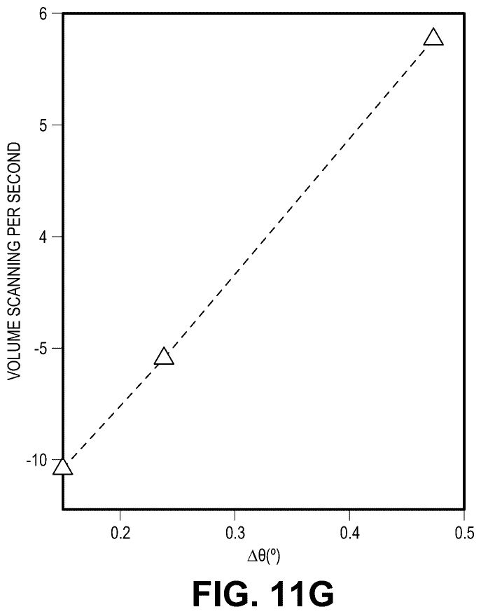

FIG. 11 illustrates global optimization of the TRUS-rSAF imaging performance. (a) Field-II wire-target simulation data at different Δθ (FOV=[0°, 66.14°]). The dots in FIG. 11(a) indicate the first grating lobe positions; (b) Definition of grating-lobe-rejected TRUS (TRUS-GLR) imaging having the first grating lobe position out of outmost plane; (c) (Left) Radial profiles at different depth. (Right) Corresponding grating lobe intensity profiles subtracted by TRUS-GLR. The heavy marks in the right column of figures in FIG. 11(c) indicate the first grating lobe positions (d) 2-D cross-correlation between the PSF of TRUS-GLR and others at 10 mm. (e) Full-width-half-maximum (FWHM) over imaging depth. (f) SNR difference between TRUS-rSAF and TRUS-REF over imaging depth. (g) Estimated volume rate at different Δθ. (h) Field-II wire-target simulation data of TRUS-REF, TRUS-rSAF, and TRUS-GLR.

FIG. 11e shows the TRUS-rSAF imaging with the optimal configuration (d_{VS}=5 mm, h=7 mm, and r=15 mm) and Δθ at 0.4724° resulting in the {46.67, −11.15, 15.65, 46.75, 54.85, 52.79, 51.00}-% narrower FWHMs at {10, 20, 30, 40, 50, 60, 70}-mm depths compared to those by the TRUS-REF imaging. Different Δθ did not produce noticeable FWHM changes. To validate the clinical efficacy of the volumetric TRUS-rSAF imaging, we compared the resultant radial imaging resolution at each depth with those by the TRUS imaging using an in-plane micro-convex array (BPC8-4/10) located perpendicular to the linear array for volumetric imaging in our clinical bi-planar TRUS transducer. The specifications of the BPC8-4/10 are as follows: the number of channels, 128; 6 MHz, center frequency; fractional bandwidth, 60%; element pitch, 0.21 mm; element height, 7 mm; the radius of curvature, 10 mm; f-number in the transmit aperture, 3; transmit focusing was at 30 mm around the middle of imaging depth.

We tested a different number of active channels of the microconvex array (BPC8-4/10), as the divergence of the convex transducer configuration limits the effective aperture of the microconvex TRUS imaging. Results of quantitative measurement of full-widths-half-maximum (FWHMs) of the target cross-section reconstructed by the TRUS-REF method of microconvex array and TRUS-rSAF method using linear array are depicted in FIG. 12 and shown in Table 1. FIG. 12a depicts a transducer device 1200, consistent with this disclosure, using in-plane microconvex array coordination. FIG. 12b depicts wire target images with different effective aperture widths of the microconvex array.

In particular, FIG. 12b shows the wire targets over depth (10-70 mm in 10-mm intervals), and Table 1 below, summarizes the quantitative measurements of their FWHMs. Fractional improvement in Table 1 was calculated with a ratio between the optimal TRUS-REF and TRUS-rSAF methods, i.e., (1−TRUS-rSAF/TRUS-REF)×100). The results indicate that the 64-channel micro-convex TRUS-REF imaging is optimal with microconvex array, but the TRUS-rSAF image showed higher spatial resolution at most imaging depths.

TABLE 1

| | | Target depth for FWHM measurement | | | | | | |
|---|---|---|---|---|---|---|---|---|
| | | 1 cm | 2 cm | 3 cm | 4 cm | 5 cm | 6 cm | 7 cm |
| Microconvex | #Effective channels = 32 | 1.05 | 1.47 | 1.73 | 2.24 | 2.91 | 3.68 | 4.54 |
| TRUS-REF | #Effective channels = 64 | 1.08 | 1.40 | 1.66 | 2.19 | 2.76 | 3.34 | 3.96 |
| | #Effective channels = 96 | 1.18 | 1.52 | 1.67 | 2.23 | 2.85 | 3.47 | 4.12 |
| | #Effective channels = 128 | 1.40 | 1.80 | 1.69 | 2.32 | 3.03 | 3.77 | 4.55 |

| | | | | | | | |
|---|---|---|---|---|---|---|---|
| TRUS-rSAF (N̅_̅{̅s̅y̅n̅}̅ = 65) | 0.51 | 1.42 | 1.57 | 1.66 | 1.81 | 2.36 | 2.98 |
| Fractional improvement (%) | 52.78 | −1.43 | 5.42 | 24.20 | 34.42 | 29.34 | 24.75 |

Encouragingly, the TRUS-rSAF imaging provided the spatial resolution even higher than the in-plane microconvex TRUS imaging with 64 effective transmittance/reception channels, as shown in FIG. 11e: {53.26, −1.08, 4.92, 23.93, 34.64, 29.31, 24.78}-% narrower FWHMs at {10, 20, 30, 40, 50, 60, 70}-mm depths.

However, given the current imaging configuration (i.e., Δθ=0.4724°), the TRUS-rSAF image presented noticeable grating lobe artifacts due to coarse radial sampling (FIG. 10a). In addition, low SNR at deep imaging depths, primarily due to the diverging beam field designed to secure the wider synthetic window, should also be addressed. The analytical description of the TRUS-rSAF method (Eq. 13) allows the further optimization of the grating lobe positions and N̅_̅{̅s̅y̅n̅}̅, while preserving αₘₐₓ to secure the highest spatial resolution. The corresponding change in volume scanning rate will also be analyzed.

### Grating Lobe

FIG. 11a shows the grating lobe positions in the optimized TRUS-rSAF imaging at Δθ=0.4724°/{1.00, 1.25, 1.50, 1.75, 2.00, 3.02} (i.e., {0.4724°, 0.3779°, 0.3149°, 0.2699°, 0.2362°, 0.1564°, respectively}). We identified that the original Δθ at 0.4724° presented significant grating lobe artifacts, but its positions shifted far from the target with the finer Δθ. The dots in FIG. 11(a) indicate the theoretical grating lobe positions at each target depth, as illustrated in Eq. 13-2, and they were well matched with the radial profiles of the wire target signal, as shown in FIG. 11c. Therefore, the results validate the analytical approach as a theoretical foundation to design an effective Δθ control to alleviate the grating lobe artifacts.

Consistent with the current disclosure, one can set a performance benchmark by designing the first grating lobe positions out of the −20-dB radial beam profile of the outmost transverse plane in the synthetic window, by which the grating lobe artifacts can be effectively rejected (TRUS-GLR, FIG. 11b). One can set a strict design criterion to reject the grating lobe artifacts at the near depth, i.e., 10 mm imaging depth. Grating lobes in the deeper depth will be suppressed, following the proportional relationship of the grating lobe positions to imaging depth z_{f} in Eq. 13-2. FIG. 11b shows the single-sided radial beam profiles of the center plane and the outmost plane. The condition defines the corresponding Δθ at 0.1564°, producing the grating lobe positions at 40.96°. The corresponding B-mode image of the TRUS-GLR method successfully rejected grating lobe artifacts compared to the original design with Δθ at 0.4724°, as demonstrated in FIG. 11a. In addition, 2-D cross-correlation coefficients measured between point-spread functions (PSFs) of TRUS-GLR and other TRUS-rSAF images at 10-mm depth presented a noticeable drop from Δθ=0.2699°, suggesting grating lobe artifacts (FIG. 11d).

### Signal-to-Noise Ratio

As discussed in the previous sections, the SNR of the TRUS-rSAF imaging is lowered by the diverging acoustic transmission but can be compensated by increasing N̅_̅{̅s̅y̅n̅}̅. FIG. 11f presents the SNR difference between TRUS-rSAF and -REF imaging from the wire targets over imaging depths. Note that the change of Δθ did not affect the spatial resolution by fixing αₘₐₓ (i.e., θₘₐₓ=30.23° with N̅_̅{̅s̅y̅n̅}̅: {65, 81, 97, 113, 129, 195} at Δθ: {0.4724°, 0.3779°, 0.3149°, 0.2699°, 0.2362°, 0.1564°}).

One can test a design criterion to provide SNR comparable to that in the TRUS-REF imaging, by which the fundamental expectation in modern clinical TRUS diagnosis can be met. If one aims to have comparable or higher SNR at the entire depth range, the minimal Δθ should be 0.2362°. On the other hand, one may care more about deep tissue at 60 mm, given that the superficial region has less acoustic attenuation. In this case, the criterion is already achieved in the original TRUS-rSAF imaging specification of Δθ=0.4724°.

### Volume Scanning Rate

Although reducing Δθ is effective in alleviating grating lobe artifacts and enhancing SNR, it presents a drawback in volume scanning rate. To illustrate, we first estimated a scanning time for the conventional imaging specifications effective for both TRUS-REF and TRUS-rSAF methods: 5-cm imaging depth; 5-plane-wave angle compounding in lateral dimension; 280 scanlines at Δθ of 0.4724°, constructing FOV over [−66.14°, 66.14°]; θₘₐₓ of 30.23° (i.e., N̅_̅{̅s̅y̅n̅}̅ at 65). Note that mechanical and electrical transition times were not considered for a more straightforward presentation. The estimation starts from the round-trip time duration for the imaging depth (i.e., 6.49 μsec/cm acoustic propagation speed×5 cm×2=64.94 usec) plus a redundant duration between radial planes (assumed to be 20 μsec), multiplied by the number of lateral compounding events (84.94 μsec×five compounding events=424.70 μsec). These parameters lead to the total volume scanning time with the number of radial planes composing the volume (i.e., 424.70 μsec×280 planes=118.92 msec). In this case, a volume scanning rate is 8.41 volume/second.

On the other hand, our SNR optimization of the TRUS-rSAF method required the minimal Δθ at 0.2362° to ultimately outperform the TRUS-REF method (FIG. 11f). In this case, the volume scanning time will be 237.82 msec, leading to a 50% reduction in the imaging rate (i.e., 4.20 volume/second).

One may pursue extreme optimization for a complete rejection of grating lobe artifacts. The TRUS-GLR imaging suggests Δθ at 0.1564° for the effective rejection starting from near depth (>10 mm). In this case, the volume scanning times will be 359.18 msec, 302.04% longer than the original specifications (i.e., 118.92 msec). The corresponding volume scanning rate is 2.78 volume/second.

Given the scenarios among multiple metrics (i.e., SNR, volume scanning rate, and grating lobe), users of the TRUS-rSAF imaging must prioritize their imaging specifications when determining Δθ in the TRUS-rSAF imaging.

### Tissue-Mimicking Simulation

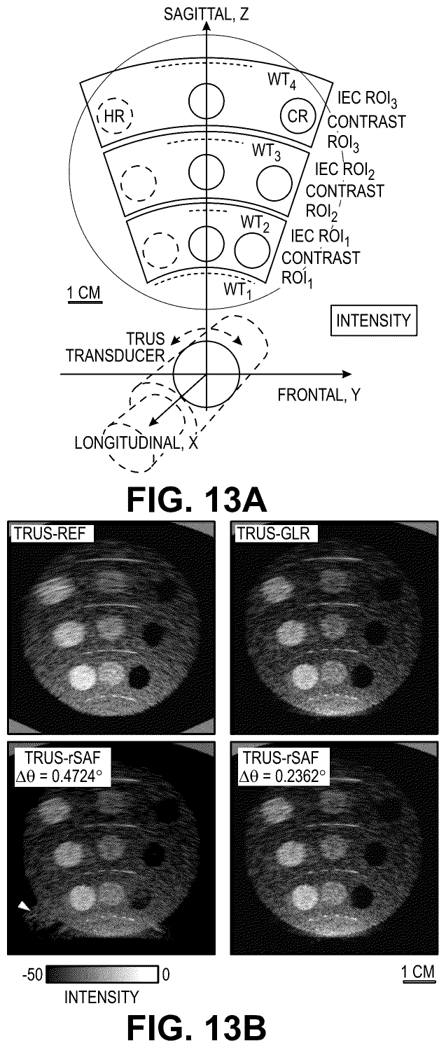

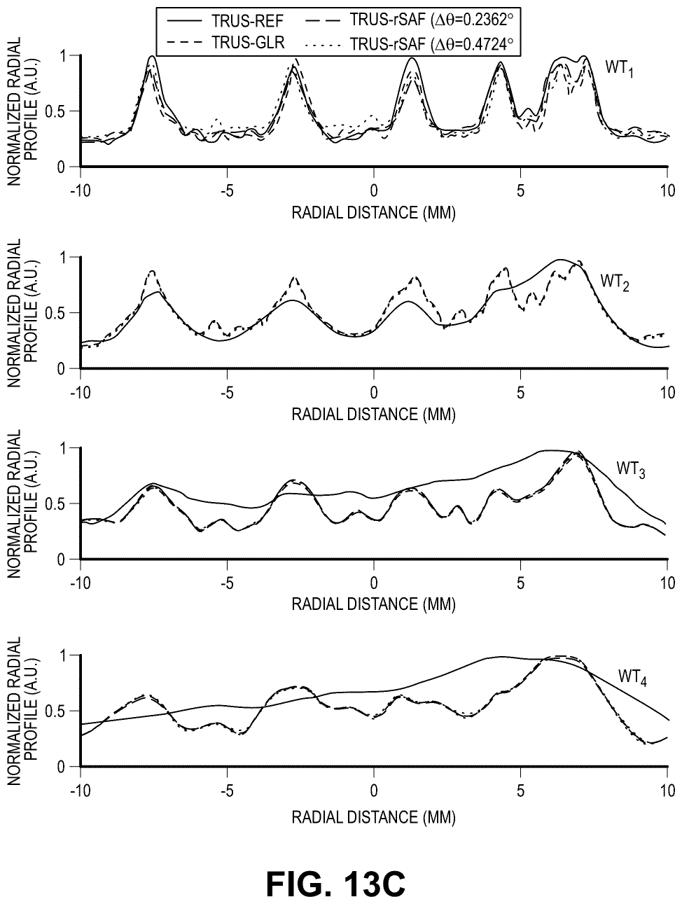

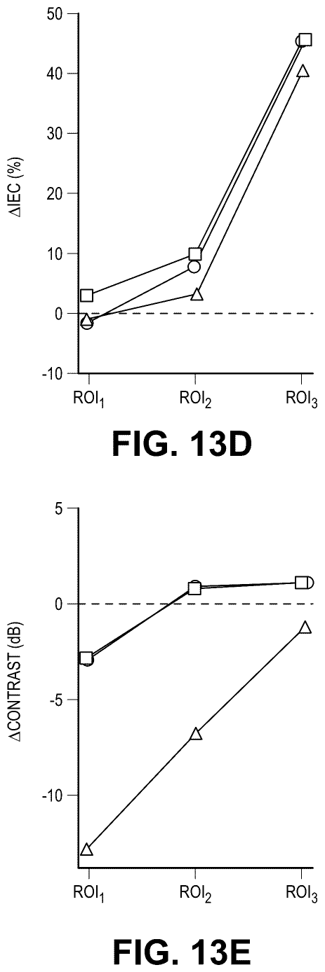

FIG. 13 depicts evaluation of TRUS imaging simulation. (a) Geometrical illustration of the prostate-mimicking phantom. HR: Hyperechoic region. CR: Cyst region. WT: Wire target. (b) TRUS images of simulated prostate imaging scenario. White dashed-line boxes represent the ROIs centered at 20-, 35-, and 50-mm, for measuring IEC (c) Normalized 1-D profiles of WT₁ (12.5 mm), WT₂ (27.5 mm), WT₃ (42.5 mm), and WT₄ (57.5 mm). (d), (e)

A prostate tissue-mimicking Field-II simulation was performed to evaluate the functional performance of the TRUS-rSAF framework. FIG. 13a shows the ground-truth field definition with wire targets (WT), hyperechoic mass (HR), and hypoechoic cyst (CR). FIG. 13b demonstrates the simulated B-mode images of TRUS-REF and TRUS-rSAF with different scanline intervals: Δθ=0.4724°, 0.2362° and 0.1564° (TRUS-GLR). In visual assessment, apparent enhancement in spatial resolution was identified in the TRUS-GLR imaging. Relative improvements over the TRUS-REF imaging were evident in the deeper imaging depth. FIG. 13c shows the 1-D radial profiles of the wire targets (WT₁₋₄), agreeing with the expectation from FIG. 11e—The TRUS-rSAF method well resolved <3-mm distance in entire imaging depth regardless of Δθ. Otherwise, the TRUS-REF imaging failed to resolve the 5-mm distance between targets from 50-mm depth. On the other hand, a significant amount of grating lobe artifacts was found in the TRUS-rSAF method when Δθ=0.4724° as anticipated. The TRUS-rSAF with the optimized Δθ at 0.2362° showed the image quality comparable to that in the TRUS-GLR imaging with effective suppression of the grating lobe artifacts.

Quantitative evaluations were performed to validate the visual observations. The Contrast between the hyperechoic and hypoechoic targets in FIG. 13e indicated {30.08, 27.24, 25.12} dB in the TRUS-REF imaging at ROI₁₋₃. On the other hand, the TRUS-rSAF imaging showed {17.27, 20.32, 23.78} dB, {27.14, 27.92, 26.09} dB, and {27.20, 27.92, 26.11} dB at {ROI₁, ROI₂, ROI₃} when Δθ=0.4724°, 0.2362°, and 0.1564°, respectively. Notably, the TRUS-rSAF imaging at Δθ=0.4724° presented a low Contrast value due to severe grating lobe artifacts. Otherwise, the TRUS-GLR (Δθ=0.1564°) and the optimized TRUS-rSAF imaging (Δθ=0.2362°) successfully suppressed the grating lobe artifacts, resulting in the higher Contrast values than the TRUS-REF imaging in ROI₂₋₃. The TRUS-REF imaging provided the higher Contrast value at ROI₁ due to the lower side lobe artifacts by tightly-focused transmittance/reception event at 20 mm, but the lower spatial resolution at other imaging depths should be reminded (FIGS. 11e and 13c).

The IEC measurements at different depth range in FIG. 13d indicated the significant increase of visual information due to the TRUS-rSAF imaging. The IEC ROIs centered at {20, 35, 50}-mm depths (i.e., ROI₁, ROI₂, and ROI₃) were {2.21, 0.74, 0.25} e⁻² for the TRUS-REF method; {2.18, 0.76, 0.36} e⁻², {2.18, 0.80, 0.37} e⁻², and {2.28, 0.81, 0.37} e⁻² for the TRUS-rSAF method when Δθ=0.4724°, 0.2362° and 0.1564°. The corresponding fractional improvements over the TRUS-REF imaging were {−1.32, −1.31, 2.99} % in ROI₁, {2.59, 7.47, 9.54} % in ROI₂, and {40.48, 44.62, 45.43} % in ROI₃ for Δθ={0.4724°, 0.2362°, 0.1564°}, respectively. The results indicate that the TRUS-rSAF and TRUS-REF imaging delivers comparable amount of visual information at 20-mm depth, but the TRUS-rSAF method clearly improves the image quality throughout the image.

### 3-D Simulation

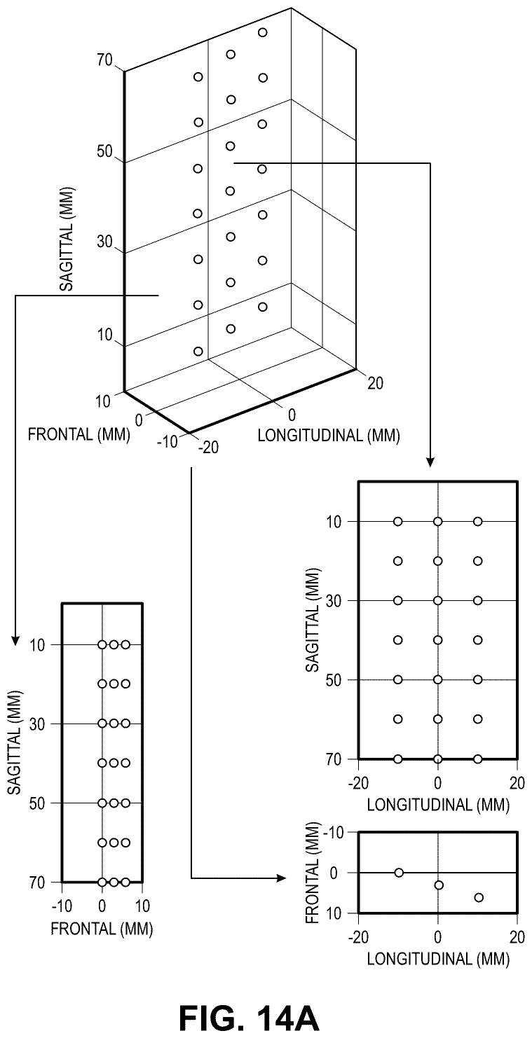

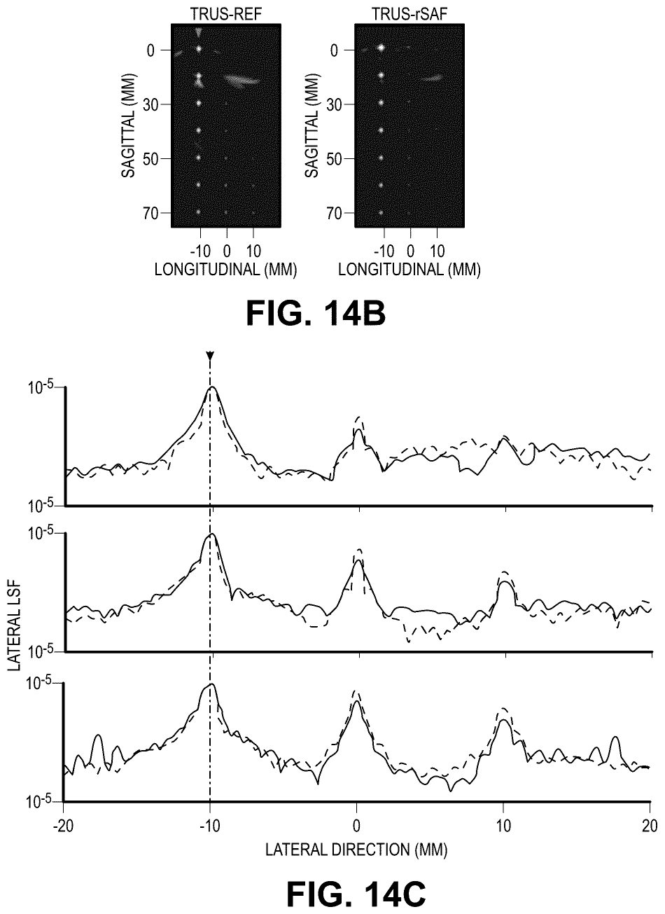

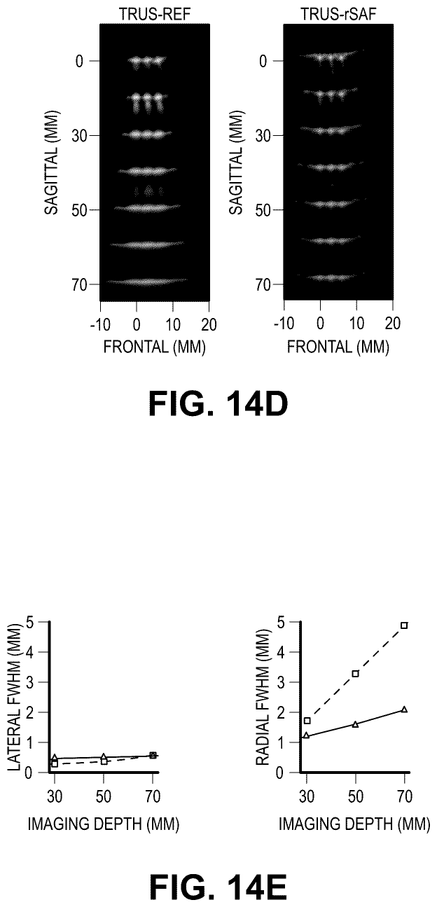

FIG. 14 depicts a 3-D imaging simulation. (a) Wire target locations in transverse-coronal-sagittal planes. (b) The TRUS-REF and TRUS-rSAF images in the transverse-sagittal dimension. (c) Line spread function (LSF) over the sagittal dimension. The dashed vertical arrow indicates the on-axis imaging plane at 0° scanning angle. (d) The transverse-coronal planes. (e) Sagittal and coronal FWHMs.

The analytical models of beamforming in two orthogonal dimensions (i.e., transverse and sagittal planes) are independent in defining theoretical spatial resolution and grating lobe positions for each dimension. Based on this, our simulation was performed in 2-D. However, there should be crosstalk in practical imaging circumstances, depending on the given transducer design, target configuration, and spatial resolution. Herein, we evaluate the practical impact of the TRUS-rSAF method using optimized critical parameters on 3-D imaging performance.

Volumetric imaging simulation was conducted with three columns and rows of point targets: {−10, 0, 10} mm in longitudinal direction and {0, 3, 6} mm in frontal direction (FIG. 14a). The point targets were repeated from 10 mm to 70 mm with a 10-mm interval on the sagittal axis. Each lateral scanning sequence consists of 31 plane-wave transmission and reception events using the entire lateral aperture of the TRUS array transducer with a range of steering from −15° to 15° at 1° interval. Depth-dependent signal processing techniques (e.g., apodization and dynamic aperture) were not applied to evaluate each method's raw TRUS imaging performance. Each lateral scanning sequence consists of multiple plane-wave transmittance and synthesis to generate a radial plane at each scanning angle. This sequence was repeated in the radial domain to scan the radial FOV from −65° to 65° with variable radial scanning interval Δθ. Note that the VS in the elevation direction of the linear array was uniform in the lateral direction and did not affect the lateral beamforming process. In the TRUS-rSAF method, multiple radial planes are synthesized with appropriate compensation of synthetic time delay at each pixel position. The TRUS-REF method only took a single radial plane obtained at the target scanning angle. The envelope of the volumetric RF data is detected by bandpass-filtered Hilbert transform (3.9-9.1 MHz), and a digital scan converter (DSC) produces 3-D TRUS image data. The target radial plane was selected at 0° scanning angle. The transverse plane was presented with the maximum intensity projection (MIP) map in the longitudinal direction. Our quantitative metrics were how much off-axis point target intensities could be suppressed in the sagittal plane and FWHMs in the transverse plane.

FIGS. 14b,d show sagittal and transverse imaging planes reconstructed by the TRUS-REF and TRUS-rSAF methods. In the sagittal plane, the substantial off-axis intensity from 0 mm and 10 mm lateral columns were identified in the TRUS-REF image primarily due to limited radial resolution, as presented in FIG. 11e. The TRUS-rSAF method also showed off-axis interferences but less intensity in deep tissue and alleviated lateral grating lobe artifacts due to radial synthesis. FIG. 14c presents lateral beam profiles at {30, 50, 70} mm imaging depths. Compared to those at on-axis intensities at −10-mm lateral column, the TRUS-rSAF method showed off-axis target of {1.78, 7.66, 16.26} % at 0-mm lateral column and {0.70, 0.99, 2.82} % at 10-mm lateral column in {30, 50, 70} mm imaging depths, respectively. On the other hand, the TRUS-REF method showed substantial interferences from the off-axis targets: {4.94, 20.06, 51.75} % and {0.79, 2.42, 9.22} % at {30, 50, 70} mm imaging depths, respectively, of those at on-axis intensities at −10 mm longitudinal column. In shallower imaging depths at 10 and 20 mm, higher off-axis intensities in the TRUS-rSAF method were identified, but there was another advantage of reducing lateral grating lobe artifacts with the radial synthesis in the depth range. FIG. 14e shows the spatial resolution measured in sagittal and transverse planes of the TRUS-REF and TRUS-rSAF images. Lateral FWHM at 0° scanning angle were {0.47, 0.48, 0.57} mm and {0.30, 0.36, 0.57} mm in the TRUS-rSAF and TRUS-REF methods at {30, 50, 70} mm imaging depths, respectively. The TRUS-rSAF method showed slightly broadened FWHM, presumably due to the changes in TRUS array design, but both methods successfully provided spatial resolution below 0.5 mm. Any other combinations of the critical design parameters (i.e., d_{VS}, h, and r) will likely produce a lateral FWHM between them, as the TRUS-REF and TRUS-rSAF methods represent highly diversified cases. In the transverse plane at the 0-mm frontal position, the same results were found as already presented in FIG. 11, showing significant improvements in radial FWHM by the TRUS-rSAF method than those by the TRUS-REF method with the fractional improvement of {25.61, 51.23, 57.73} % at {30, 50, 70} mm, respectively.

### DISCUSSIONS AND CONCLUSION

The above analysis demonstrates the novel analytical design strategy and optimization workflow of the TRUS-rSAF method to secure unprecedented volumetric imaging quality. The closed-form analytical model identified the critical parameters (i.e., d_{VS}, h, and r), affecting the TRUS-rSAF imaging spatial resolution and grating lobe positions, as theorized in Eqs. 13-1 and 13-2. Here, adding lens focusing property to the analytical solution will be a limiting factor of the integral in the a domain in our analytical development, narrowing the radial synthetic window. However, we wanted the analytical description of the TRUS-rSAF method to be a theoretical platform to investigate different SAF scenarios. This objective was achieved by assuming omni-directional acoustic propagation while having pₛ(α) as a variable synthetic window function to investigate different transmit schemes on a user's own. For example, one may try to build a TRUS array with minimal h to maximize the acoustic divergence (i.e., −90°−90°), leading to the broadest possible radial synthetic window. However, there would be a critical drawback of reduced acoustic intensity, which will lower SNR in deep imaging depth. Moreover, the fabrication of such a TRUS array transducer will be highly challenging. The VS approach with the elevation focusing lens secures translational practicality, providing wide-enough pₛ(α) and transmit power in deep tissue, and pₛ(α) is decided by testing a different number of radial planes for synthesis to minimize the FWHM.

The perspectives obtained from the analytical approach led to the solid optimization workflow, resulting in significantly superior spatial resolution compared to the volumetric TRUS-REF imaging. The analytical solution derives spatial resolution and grating lobe positions by observing continuous-wave interactions from different transmit source positions. One might be concerned about the monochromatic analysis at the center frequency of the TRUS array transducer (6.5 MHz), which will make it challenging to quantify its correlation to practical simulation results that consider pulsed acoustic transmission with broad bandwidth. However, our simulation showed ‘qualitative’ agreement by the Eq. (13-1), providing spatial resolution proportional to r and αₘₐₓ and accurate localization of the first grating lobe positions, enough to be a design framework to optimize the TRUS imaging performance. The spatial resolution-oriented optimization let the TRUS-rSAF method even outperform the in-plane micro-convex TRUS imaging of a transverse plane (FIG. 11), which is a promising advance to innovate the pelvic diagnosis.

On the other hand, the impacts of the optimization on the signal sensitivity and interferences were balanced for practical image quality enhancement. From the analytical model of the TRUS-rSAF imaging, we propose an essential role of the Δθ parameter to define the SNR and the first grating lobe positions. Specific optimization criteria were to sustain the SNR level of the TRUS-REF imaging while completely removing the grating lobe artifacts. Moreover, a prostate mimicking phantom was simulated to evaluate the clinical effectiveness of the optimized TRUS-rSAF method. In addition to the higher spatial resolution compared to the TRUS-REF method, TRUS-rSAF imaging delivered more image information than the TRUS-REF method with higher Contrast and IEC metrics. On the other hand, a clinically effective volume scanning rate is implementable at the user's preference on imaging quality when compared to a clinical 3-D TRUS imaging: 2-8 Hz in FIG. 11g vs. 3-6 Hz (Fenster et al., 2011; Fenster & Downey, 1999).

In terms of computing time, our data processing time for a single volume reconstruction by the TRUS-REF and TRUS-rSAF methods took 12.4 and 18.5 hours under single-core processing with a personal laptop (2.6 GHz Intel Core i7 8850H, 16 GB RAM, MATLAB 2020b). However, they are just technical numbers and do not reflect what should be expected in a clinical device. The SAF method has been widely used in clinical real-time US imaging platforms and has been marginally handled by modern GPU-accelerated computing units and efficient beamformer architectures (Jensen et al., 2013; Park et al., 2010; Stuart et al., 2021; Yiu et al., 2011). There is no technological hurdle to secure a clinically appropriate level of temporal resolution.

3-D imaging simulation also brought attention to the benefits of the TRUS-rSAF method to obtain lower off-axis interferences and lateral grating lobe artifacts than those in the TRUS-REF method. Experimental validation is limited at this research stage, as our TRUS-rSAF method necessitates the extensive revision of the current clinical TRUS array transducer, which is currently not accessible to us. We performed a limited phantom study evaluating the sore effect of r changes using a clinical TRUS array (BPL9-5/55) maneuvered by two translational stages and one rotational stage. We achieved a high positive correlation between simulation and real-world data.

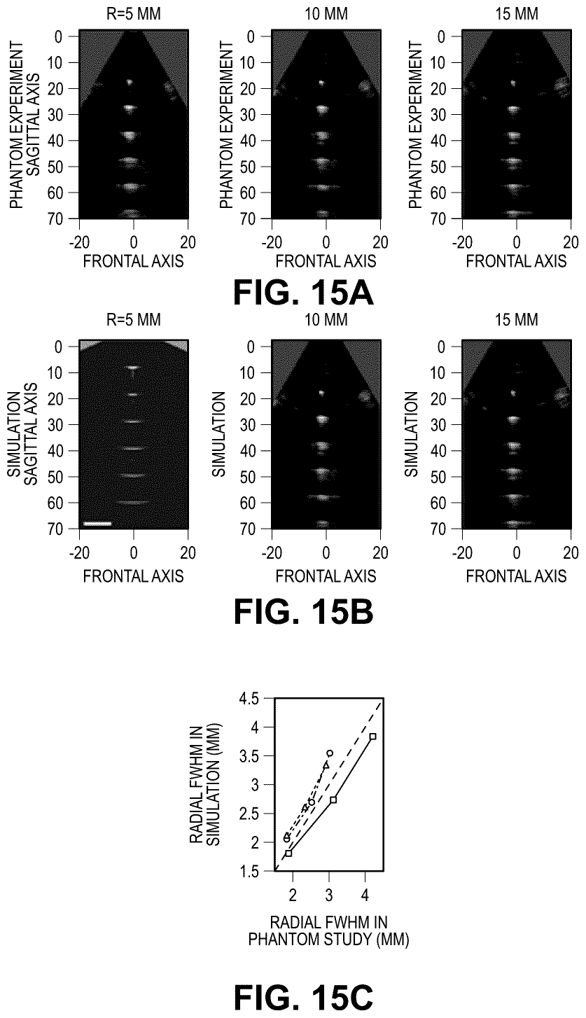

We correlated the simulation results with a practical phantom experiment. We have performed a wire target experiment using a clinically available TRUS transducer (BPL9-5/55; dys=20 mm, r=10 mm, and h=5 mm) to sorely evaluate the effect of r (i.e., {5, 10, 15} mm). Virtual scanning trajectories were made by two translational (MTS50-Z8, Thorlabs, Inc.) and one rotational stage (PRMIZ8, Thorlabs, Inc.). In the phantom experiment, point targets were posed over the depth range from 20 mm to 70 mm at 10-mm intervals. Results of this phantom experiment using a clinical TRUS array are depicted in FIG. 15. FIG. 15a depicts wire target images in the phantom experiment and FIG. 15b depicts simulation.

Due to the narrow elevation slice thickness of the clinical TRUS array, our optimization yielded unchanged N̅_̅{̅s̅y̅n̅}̅ at 7 for all three r setups, but the numerical calculation of the radial synthetic windows gave {0.29, 0.58, 0.87} mm for r={5, 10, 15} mm, respectively. The FWHMs measured at {30, 50, 70} mm were {1.87, 3.12, 4.23} mm, {1.83, 2.52, 3.03} mm, and {1.82, 2.33, 2.93} mm, respectively, when r=5, 10, and 15 mm (FIG. 15a). The trend in general agrees with the simulation outcomes: {1.80, 2.73, 3.85} mm, {2.04, 2.69, 3.56} mm, and {2.11, 2.61, 3.34} mm. FIG. 15c shows the correlation between radial FWHMs measured in the phantom experiment and simulation. The dotted line indicates the unity line. A high positive correlation was found. The slight differences between phantom and simulation results should be due to the difference in frequency bandwidth for back-end processing. The simulation study had strictly bandpass-filtered data between 3.9-9.1 MHz, −6-dB bandwidth of the BPL9-5 probe, while the experimental study uses the entire frequency band beyond −6-dB of normalized acoustic frequency response.

However, regardless of the encouraging results, an implication of the data is still insufficient to present the full benefits of our TRUS-rSAF method. Therefore, we will present complete experimental evidence of the TRUS-rSAF imaging enhancements in our following study.

Image processing to further suppress grating lobe artifacts

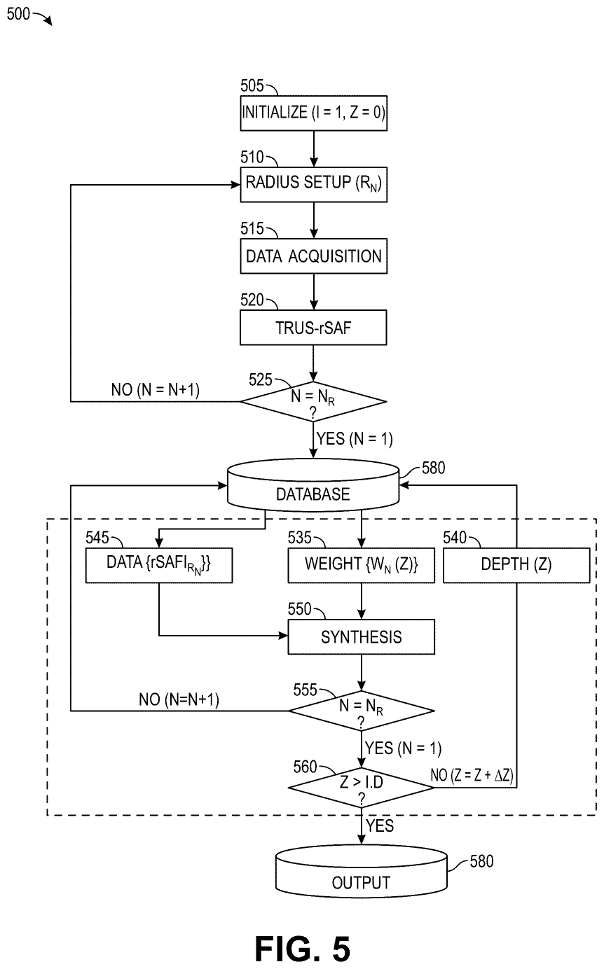

FIG. 5 depicts a method for further improving imaging quality beyond that obtained with straightforward implementation based on analytical rSAF description consistent with the current disclosure. In FIG. 5, Nᵣ is the number of radius used for scanning, I.D: Imaging depth

One might synthesize multiple images obtained with multiple radius of the transformable transducer head. As an example, FIG. 5 shows a flowchart that describes the data acquisition and synthesizing process. the shallower imaging depth can be reconstructed by incorporating more information from the image with smaller radius and/or finer scanning angle interval Δθ, providing less grating lobe artifact (Eq. 13-2). On the other hand, deeper imaging depth can be reconstructed with larger radius to focus on improving the spatial resolution.

References cited herein are included in the Appendix.

Other embodiments of the invention will be apparent to those skilled in the art from consideration of the specification and practice of the embodiment disclosed herein. It is intended that the specification and examples be considered as exemplary only, with a true scope and spirit of the invention being indicated by the enumerated claims.

What is claimed is:

1. A probe, comprising:
a multiradius transducer; and
a support comprising a primary axis;
wherein the multiradius transducer comprises:
a proximal region;
a distal region; and
a transducer array;
wherein the proximal region is adjacent the support, and the multiradius transducer terminates at the distal region;
wherein, in a first radius mode, the multiradius transducer exhibits a first surface extending from the proximal region to a distal surface, the distal surface being in the distal region, said first surface characterized by a length dimension approximately parallel to said primary axis and said first surface characterized by a first radius extending perpendicular to said length dimension;
wherein said transducer array is situated on said first surface between said proximal region and said distal surface; and
wherein, in a second radius mode, the multiradius transducer exhibits a second surface extending from the proximal region to a second distal surface in said distal region, said second surface characterized by a second radius extending perpendicular to said length dimension; and
wherein said transducer array is situated on said second surface between said proximal region and said distal region; and
wherein said second radius is greater than said first radius.

2. The probe of claim 1, wherein said transducer array is configured for acoustic reception only.

3. The probe of claim 1,
wherein said probe is an ultrasound and photoacoustic probe, and
wherein said transducer array is configured for ultrasound and/or photoacoustic imaging.

4. The ultrasound and photoacoustic probe of claim 3,
wherein external light and/or acoustic transmit systems focally or broadly deliver the energy.

5. The ultrasound and photoacoustic probe of claim 3,
wherein the multiradius transducer in either first radius mode or second radius mode is configured to scan a limited volume, said limited volume determined by the amount of light or acoustic energy received by the limited volume.

6. The ultrasound and photoacoustic probe of claim 3,
wherein the multiradius transducer identifies a plurality of positions in a scanning volume associated with a plurality of optical and/or acoustic energy transmittance values, said identification being based on acoustic data analysis or external tracking and position registration.

7. The ultrasound and photoacoustic probe of claim 3,
wherein, when the multiradius transducer is in first radius mode, said transducer array situated on said first surface extends linearly along the first surface between said proximal region and said distal surface; and
wherein, when the multiradius transducer is in second radius mode, said transducer array situated on said second surface extends linearly along the second surface between said proximal region and said distal region.

8. The ultrasound and photoacoustic probe of claim 7, further comprising:
a second transducer array;
wherein, when the multiradius transducer is in first radius mode, said second transducer array is situated on said first surface between said transducer array and said distal surface; and
wherein, when the multiradius transducer is in second radius mode, said second transducer array is situated on said second surface between said transducer array and said distal region.

9. The ultrasound and photoacoustic probe of claim 3,
wherein, when the multiradius transducer is in first radius mode, said transducer array situated on said first surface extends in a convex orientation at the first radius along the first surface between said proximal region and said distal surface; and
wherein, when the multiradius transducer is in second radius mode, said transducer array situated on said second surface extends in the convex orientation at the second radius along the second surface between said proximal region and said distal region.

10. The ultrasound and photoacoustic probe of claim 3,
wherein the first surface exhibits a generally cylindrical shape.

11. The ultrasound and photoacoustic probe of claim 3,
wherein the first distal surface exhibits a hemispherical shape.

12. The ultrasound and photoacoustic probe of claim 3,
wherein the multiradius transducer further comprises a multiaxis support, said multiaxis support comprising a second axis;
wherein, in the first radius mode, the second axis is approximately parallel with the primary axes and exhibits a first radial offset from the primary axis;
wherein, in the second radius mode, the second axis is approximately parallel with the primary axis and exhibits a second radial offset from the primary axis; and
wherein the second radial offset is greater than the first radial offset.

13. The ultrasound and photoacoustic probe of claim 12,
wherein the first radial offset is approximately zero.

14. The ultrasound and photoacoustic probe of claim 3,
wherein the transducer array is configured for rotational scanning.

15. An ultrasound and photoacoustic probe, comprising:
a multiradius transducer; and
a support comprising a primary axis;
wherein the multiradius transducer comprises:
a proximal region;
a distal region; and
a transducer array and a light fiber bundle for ultrasound and/or photoacoustic imaging;
wherein the proximal region is adjacent the support, and the multiradius transducer terminates at the distal region;
wherein, in a first radius mode, the multiradius transducer exhibits a first surface extending from the proximal region to a first distal surface, the first distal surface being in the distal region, said first surface characterized by a length dimension approximately parallel to said primary axis and said first surface characterized by a first radius extending perpendicular to said length dimension;
wherein said transducer array is situated on said first distal surface; and
wherein, in a second radius mode, the multiradius transducer exhibits a second surface extending from the proximal region to a second distal surface in said distal region, said second surface characterized by a second radius extending perpendicular to said length dimension; and
wherein said transducer array is situated on said second distal surface; and
wherein said second radius is greater than said first radius.

16. A method of ultrasound and photoacoustic scanning, comprising:
scanning a target volume using the ultrasound and photoacoustic probe of claim 3 to acquire target volume data;
wherein said target volume comprises a plurality of volumes, and wherein said scanning includes repetitively scanning said plurality of volumes.

17. The method of ultrasound and photoacoustic scanning of claim 16,
wherein said scanning comprises rotating said ultrasound and photoacoustic probe generally about the primary axis.

18. The method of ultrasound and photoacoustic scanning of claim 17, further comprising identifying and rejecting a plurality of grating lobe artifacts in said acquired target data.

19. The ultrasound and photoacoustic probe of claim 3,
wherein said transducer array further comprises a light fiber bundle.

20. The ultrasound and photoacoustic probe of claim 8,
wherein said second transducer array further comprises a light fiber bundle.

21. The method of ultrasound and photoacoustic scanning of claim 16 further comprising acquiring data determining a maximal radius in second radius mode, and
limiting said second radius to the determined maximal radius prevent tissue damage due to excessive expansion of the probe.

22. The method of ultrasound and photoacoustic scanning of claim 21
wherein said data determining said maximal radius in second radius mode is acquired using image and/or pressure sensors.
